(19) United States
(12) Patent Application Publication
(10) Pub. No.: US 2014/0127169 A1
(43) Pub. Date: May 8, 2014

(54) INDUCED PRESOMITIC MESODERM (IPSM) CELLS AND THEIR USE

(71) Applicants: Olivier Pourquie, Illkirch (FR); Matthias Wahl, Leverkusen (FR); Jerome Chal, Illkirch (FR)

(72) Inventors: Olivier Pourquie, Illkirch (FR); Matthias Wahl, Leverkusen (FR); Jerome Chal, Illkirch (FR)

(73) Assignees: UNIVERSITE DE STRASBOURG, Strasbourg (FR); STOWERS INSTITUTE FOR MEDICAL RESEARCH, MI (US); ASSOCIATION FRANCAISE CONTRE LES MYOPATHIES, Paris Cedex 13 (FR)

(21) Appl. No.: 13/981,408

(22) PCT Filed: Jan. 24, 2012
(86) PCT No.: PCT/EP2012/051029

§ 371 Date: Jan. 6, 2014

Related U.S. Application Data
(60) Provisional application No. 61/435,489, filed on Jan. 24, 2011.

(30) Foreign Application Priority Data
Jan. 24, 2011 (EP) 11151889.0

(51) Int. Cl. A61K 35/12; C12N 5/074

(52) U.S. Cl. CPC A61K 35/35; C12N 5/0696
USPC 424/93.7; 435/377; 435/455; 435/366

(57) ABSTRACT

The invention relates to a method for reprogramming target cells to multipotent progenitor cells capable of differentiating into muscular, skeletal or dermal cell lines. In particular, the invention relates to an ex vivo method for preparing induced presomitic mesoderm (iPSM) cells, said method comprising the steps of: a) providing target cells to be reprogrammed, and, b) culturing said target cells under appropriate conditions for reprogramming said target cells into iPSM cells, wherein said appropriate conditions comprises increasing expression of at least one T-Box transcription factor in said target cells. The invention further relates to the use of said iPSM cells, for example, for regenerating skeletal, muscle, dermal and cartilage tissues.

### FIELD OF THE INVENTION

The invention relates to a method for reprogramming target cells to multipotent progenitor cells capable of differentiating into muscular, skeletal or dermal cell lines. In particular, the invention relates to an ex vivo method for preparing induced presomitic mesoderm (iPSM) cells, said method comprising the steps of:
a) providing target cells to be reprogrammed; and,
b) culturing said target cells under appropriate conditions for reprogramming said target cells into iPSM cells, wherein said appropriate conditions comprises increasing expression of at least one T-Box transcription factor in said target cells.

The invention further relates to the use of said iPSM cells, for example, for regenerating skeletal, muscle, and dermal tissues.

### BACKGROUND OF THE INVENTION

Embryonic stem (ES) cell research offers unprecedented potential for understanding fundamental developmental processes, such as lineage differentiation. Embryonic stem cell lines are derived from early embryos and are characterized by their ability to self-renew, that is, to be maintained indefinitely in a proliferative and undifferentiated state in culture. ES cells are also pluripotent, meaning they retain the capacity to differentiate into the three embryonic lineages: ectoderm, mesoderm and endoderm plus all of their derivatives (Chambers, 2004). The recent development of reprogramming technologies now allows ES-like stem cells to be generated from somatic cells, such as fibroblasts. Introduction into somatic cells of a small set of specific transcription factors—Oct4, Sox2, c-Myc, and Klf4 in the mouse (Takahashi and Yamanaka 2006) and human (Takahashi et al. 2007; Park et al. 2008b), or Oct4, Sox2, Nanog and Lin28 in human (Yu et al. 2007)—can reprogram various differentiated cell types to an ES-like stem cell state (inducible pluripotent stem cells or iPS). This strategy now allows the generation of ES-like cell lines from individual patients and, thus, offers the possibility to create highly relevant in vitro models of human genetic diseases. Such reprogrammed cell lines have already been generated from patients with a variety of diseases, such as Duchenne Muscular Dystrophy or Amyotrophic lateral sclerosis (ALS) and differentiation of the reprogrammed cells into the deficient tissue has been achieved for iPS cells from ALS patients, thus, demonstrating the feasibility of the approach (Dimos et al. 2008; Park et al. 2008a).

Whereas some lineages such as cardiac myocytes or neurons are easily generated in vitro from ES cells, differentiating skeletal muscle from ES or iPS cells has proven to be challenging. Given the promises offered by cellular replacement therapy for the cure of some muscular degenerative diseases or for orthopaedic surgery, the development of protocols for production of precursors of muscle and skeletal lineages is of key importance.

In the embryo, the muscles, and the axial skeleton of the body derive from multipotent precursors forming the presomitic mesoderm (PSM). These precursors are characterized by expression of the genes Brachyury (T), Tbx6 and Mesogenin1(Msgn1), and they mostly differentiate into skeletal muscles, dermis, skeletal lineages, as well as in a variety of other derivatives including adipocytes and endothelial cells. Transcription factors of the MyoD family have long been known to be capable of reprogramming differentiated cells (such as fibroblasts) toward a muscle fate when introduced ectopically into these cells (Weintraub et al. 1989). However, the process is rather inefficient and the reprogrammed cells have limited proliferative potential, which makes them poorly suited for regenerative medicine applications (Dinsmore et al. 1996).

The Brachyury gene is also known to be expressed during embryogenesis in the precursors of the developing skeleton and overexpression of Brachyury can convert mesenchymal cell lines to a cartilage—like tissue (Hoffmann et al. 2002; Dinser R, et al. 2009). Based on these findings, methods of inducing cartilage repair by administering a cell expressing T Box factor have also been suggested by Gazit et al (U.S. Pat. No. 6,849,255 B2). However, the described cell lines are restricted to progenitor cells of cartilage-like tissues.

Therefore, there is still a need to provide for methods for reprogramming target cells to pluripotent precursor lineages, in particular being capable of regenerating muscular, skeletal or dermal tissues.

The present invention fulfils this need by providing a method for preparing multipotent progenitor cell lines referred to as induced presomitic cells (iPSM cells), from any target cell, including differentiated target cells, said iPSM cells being then capable of giving rise to cell lineages of the muscular, skeletal or dermal tissue. The inventors have shown that fibroblasts can be reprogrammed into iPSM cells using a limited number of steps. In particular, the inventors have made the surprising finding that it is possible to obtain presomitic-like cells by the introduction of only one or two reprogramming factors. They have shown that the obtained iPSM cells can be cultured and proliferate at the undifferentiated presomitic stage indefinitely. In some advantageous embodiments, the methods of the invention allow the preparation of iPSM cells without any genetic modification of the target cells.

The invention requires overexpressing the T-Box gene Brachyury (which is expressed in the multipotent precursors of the PSM in the embryo) in target cells, which target cells may be differentiated cells such as dermal fibroblasts. The inventors have further demonstrated that cells overexpressing the T-Box gene Brachyury in differentiated target cells, such as dermal fibroblasts, can activate markers of the PSM such as Msgn1 or Tbx6, indicating that they can be effectively reprogrammed as multipotent progenitors of the PSM. These reprogrammed cells were termed iPSM and, like PSM cells, they are advantageously able to generate the muscle, skeletal and dermal lineages.

To the applicant's knowledge, the invention is the first description of a method for obtaining unlimited amounts of cells suitable for use as self-renewing progenitor cells for regenerating either muscle, skeletal or dermal tissues, therefore the invention is highly useful in particular in regenerative medicine, and will also find numerous applications in the research field.

### SUMMARY OF THE INVENTION

A first aspect of the invention relates to an ex vivo method for preparing induced presomitic mesoderm (iPSM) cells, said method comprising the steps of:
a) providing target cells to be reprogrammed, and,
b) culturing said cells under appropriate conditions for reprogramming said target cells into iPSM cells, wherein said appropriate conditions comprises increasing expression of at least one T-Box transcription factor in said cells.

Optionally, the method may further comprise a step of detecting or selecting among the cultured cells, those expressing one or more of the biomarkers characteristic of presomitic mesoderm cells.

Advantageously, said induced presomitic mesoderm cells have long-term self-renewal properties.

In a preferred embodiment, said appropriate conditions for reprogramming cells into iPSM cells further comprise inhibiting at least retinoic acid signalling in said cells.

The target cells to be reprogrammed may be selected from primary cells, differentiated cells, for example differentiated somatic cells such as fibroblasts, for example mouse or human fibroblasts. In one specific embodiment, the target cells are primary cells or fibroblasts obtained from a human patient in need of regenerative medicine. In one specific embodiment, such target cells do not include human embryonic cells.

In one embodiment, conditions for increasing expression of at least one T-Box transcription factor comprises introducing an expression vector comprising the gene encoding said T-box transcription factor into the cells for ectopic expression of the gene encoding said T-box transcription factor.

In another embodiment, conditions for increasing expression of at least one T-box transcription factor comprises the direct introduction of an effective amount of the T-box transcription factor or its precursor RNA, whether modified or not, as a reprogramming factor, into said cells.

In another embodiment, conditions for increasing expression of at least one T-box transcription factor comprises enhancing the endogenous expression of T-box transcription factor, for example by modulating Wnt, BMP and FGF signalling.

A preferred example of a T-box transcription factor which can be used as a reprogramming factor is Brachyury transcription factor.

In another embodiment, inhibition of retinoic acid signalling is achieved in the method of the invention by:
a) ectopic expression of a nucleic acid construct encoding a dominant negative retinoic acid receptor (dnRAR) in said target cells;
b) culturing the target cells in the presence of an appropriate amount of one or more inhibitors of retinoic acid receptor signalling or of retinaldehyde dehydrogenase inhibitors, or, in medium depleted in retinoids;
c) inhibiting endogenous expression of a gene involved in retinoic acid signalling in said target cells; or,
d) overexpressing proteins involved in retinoic acid catabolism such as Cyp26 in said target cells.

In a preferred embodiment of the method of the invention, the method does not involve any genetic modification of the target cells to be reprogrammed and said conditions for increasing expression of at least one T-box transcription factor comprises the direct introduction of the Brachyury transcription factor into said target cells or its precursor RNA in an amount sufficient for auto-induction of the endogenous expression of Brachyury transcription factor, and said appropriate conditions for reprogramming the target cells into iPSM cells further comprise culturing the target cells in the presence of an appropriate amount of one or more inhibitors of retinoic acid signalling.

Another aspect of the invention relates to a composition comprising iPSM cells obtainable from the methods of the invention, characterized in that at least 10%, 20%, 30%, 40%, 50%, 60%, 70%, 80% and preferably at least 90% of the cells in said composition, exhibit a high expression of a biomarker characteristic of presomitic mesoderm cells, for example, Mesogenin1 (Msgn1) gene product and/or Tbx6 gene product.

The invention further relates to the use of said iPSM cells for obtaining cell lineages of skeletal muscle, bone, cartilage and dermal tissues and in particular to the methods for preparing compositions comprising skeletal muscle, bone, cartilage or dermal cell lineages, said method comprising the steps of
(a) providing a composition comprising iPSM cells according to the invention; and,
(b) culturing said composition comprising iPSM cells under appropriate conditions for differentiation of the iPSM cells into the desired cell lineages selected among the group consisting of skeletal muscle, bone, cartilage or dermal cells.

The invention further relates to a method for preparing compositions comprising skeletal muscle cell lineages, said method comprising the steps of
(a) providing a composition comprising iPSM cells;
(b) culturing said composition comprising iPSM cells in the presence of a differentiation medium comprising at least the following components:
   (i) an extracellular matrix material, and,
   (ii) compounds activating or inhibiting the signalling pathways known to control of the differentiation of said lineages which include, but are not restricted to, retinoic acid, BMP, Hedgehog, Notch, FGF, Wnt, myostatin, insulin, PDGF, MAPK and PI3K; and,
(c) optionally culturing said composition obtained from step (b) in a second differentiation medium comprising at least one or more of the following differentiation factors bFGF, HGF, horse serum, Activin A, transferrin, EGF, insulin, LiCl, and IGF-1,
thereby obtaining a composition comprising skeletal muscle cell lineages.

In another embodiment, the present invention provides a method for preparing a composition comprising dermal cell lineages, said method comprising the steps of culturing a composition comprising iPSM cells in the presence of an efficient amount of at least one or more of the differentiation factors selected from the group consisting of BMP, Wnt, FGF, EGF, retinoic acid, and Hedgehog families of growth factors.

In another specific embodiment, the present invention provides a method for preparing a composition comprising bone or cartilage cell lineages, comprising the step of culturing a composition comprising iPSM cells in the presence of an efficient amount of at least one or more of the differentiation factors selected from the group consisting of retinoic acid, Wnt, Hedgehog, pTHRP, TGF, BMP families of growth factors, dexamethasone, ascorbic acid, vitamin D3 and b-glycerophosphate.

The invention thus provides a composition comprising muscle, bone, cartilage or dermal cell lineages derived from differentiation of iPSM cells, as obtainable by the differentiation methods described above.

The compositions of the invention described above may advantageously be used, as cell therapy product, or in regenerative medicine, in the treatment of muscle genetic disease, for example, Duchenne muscular dystrophy; in the treatment of joint or cartilage or bone damages or disorders in orthopaedic surgery, or in production of dermal tissue, for example for the cosmetic and pharmaceutical industry.

The compositions of the invention described above may be advantageously used for production of differentiated muscle, dermal, and skeletal derivatives as well as endothelial, meninges or adipocytes derivatives from healthy or disease-bearing patients for screening or for toxicology assays for the pharmaceutical and cosmetic industry.

### DETAILED DESCRIPTION OF THE INVENTION

A first aspect of the invention relates to an ex vivo method for preparing induced presomitic mesoderm (iPSM) cells, said method comprising the steps of:
a) providing target cells to be reprogrammed; and,
b) culturing said target cells under appropriate conditions for reprogramming said target cells into iPSM cells, wherein said appropriate conditions comprise increasing expression of at least one T-Box transcription factor in said target cells.

As used herein the term “induced presomitic mesoderm cells” or “iPSM” refers to cells derived from any cell type but exhibiting characteristics of embryonic cells of the presomitic mesoderm.

The iPSM cells have long term self renewal properties, e.g., they can be maintained in culture more than 6 months.

In one embodiment, the iPSM cells are further characterized by the following properties:
a) they are derived from reprogramming a target cell,
b) they express biomarkers characteristic of presomitic mesoderm cells such as Msgn1 gene, as measured for example with a gene reporter assay comprising the Msgn1 promoter, and,
c) they are multipotent cells, capable of differentiating into at least skeletal, dermis or muscle cell lineages;

The multipotency of said iPSM cells can be tested in vitro, e.g., by in vitro differentiation into skeletal, dermal or muscle cell lineages using the protocols described below, and in particular in the Examples.

The term “reprogramming” refers to the process of changing the fate of a target cell into that of a different cell type, caused by the expression of a small set of factors (or reprogramming factors) in the target cells. For example, primary fibroblasts can be reprogrammed to ES-like stem cells or induced pluripotent stem cells by expressing ectopically Oct3/4, Sox2, c-myc and Klf4 (Takahashi and Yamanaka, 2006). Fibroblast can also be reprogrammed to cardiomyocytes by overexpressing GATA4, Mef2c and Tbx5 (Direct reprogramming of fibroblasts into functional cardiomyocytes by defined factors. Ieda et al. 2010) or to neurons by overexpressing the three transcription factors Ascl1, Brn2, and Mytl1 (Vierbuchen, et al. 2010).

The term “multipotent” refers to cells that can differentiate in more than one cell lineage depending on the environmental and culture conditions. Contrary to embryonic stem cells which are pluripotent and can differentiate into all types of somatic cell lineages, the induced presomitic mesoderm cells of the present invention have limited differentiation capacity.

### The Target Cells to be Reprogrammed

The target cells to be reprogrammed in the method of the present invention are selected from mammals, and preferably from rodent, primate or human species, more preferably from mouse or human species.

In one preferred embodiment, said target cells are primary cells, including embryonic or somatic cells, for example, differentiated cells. In a related embodiment, said target cells are adult somatic cells, primary cells from adult somatic cells.

As used herein, the term “primary cells” refers to cells that are obtained from living tissue (e.g. biopsy material) and have not undergone immortalization process.

In another specific embodiment, said target cells are obtained from primary cells from blood, bone marrow, adipose tissue, skin, hair, skin appendages, internal organs such as heart, gut or liver, mesenchymal tissues, muscle, bone, cartilage or skeletal tissues.

As used herein the term “differentiated” is used to refer to a cell that is not capable of giving rise to more than one cell lineage in a natural environment.

The target cells to be reprogrammed may be obtained from existing commercial primary cells or cell lines or obtained from various tissues, for example from primary cells or reprogrammed iPS cells or their derivatives, for example from a human patient in need of regenerative treatment or from an animal model, such as, a transgenic mouse line.

Methods to obtain samples from various tissues and methods to establish primary cells are well-known in the art (see e.g. Jones and Wise, 1997). Suitable cells may also be purchased from a number of suppliers such as, for example, the American Tissue Culture Collection (ATCC) or the German Collection of Microorganisms and Cell Cutures (DSMZ).

In one preferred embodiment, said cells to be used in the present invention are fibroblast cells, for example, human or mouse fibroblasts.

The T Box Transcription Factor for Use as a Reprogramming Factor to Obtain iPSM Cells

One essential feature of the present invention is the use of a T Box transcription factor as a reprogramming factor to obtain iPSM cells.

As used herein, the term “T Box transcription factor” refers to a family of transcription factors that share the T-box domain, a 200 amino acid DNA-binding domain. The T-box family has been identified in both vertebrates and in non-vertebrates and is known to play a key role in embryonic development. Brachyury (also known as T) is the founding member of the T-box family. In one specific embodiment of the method of the invention, the Brachyury transcription factor is used as a reprogramming factor to obtain iPSM cells.

As used herein, the term “Brachyury” refers to the T-box transcription factor encoded by the T gene. Typically, the human Brachyury has the polypeptide sequence of SEQ ID NO:1 as defined in Genbank accession number NP_{—}003172. The mouse Brachyury has the polypeptide sequence of SEQ ID NO:2 as defined in Genbank accession number NP_{—}033335. The skilled person may select other Brachyury transcription factors originating from mammals, such as humans, mice, rats, cows, horses, sheep, pigs, goats, camels, antelopes, and dogs. Advantageously the skilled person may select the corresponding Brachyury transcription factor from the same species as the target cells used as starting material in the method of the invention.

As used herein, the term “Brachyury” also encompasses any functional variants of Brachyury wild type (naturally occurring) protein, provided that such functional variants retain the advantageous properties of reprogramming factor for the purpose of the present invention. In one embodiment, said functional variants are functional homologues of Brachyury having at least 60%, 80%, 90% or at least 95% identity to the most closely related known natural Brachyury polypeptide sequence, for example, to human or mouse polypeptide Brachyury of SEQ ID NO:1 or SEQ ID NO:2 respectively, and retaining substantially the same transcriptional factor activity as the related wild type protein. In another embodiment, said functional variants are fragments of Brachyury, for example, comprising at least 50, 100, 200 or 300 consecutive amino acids of a wild type Brachyury protein, and retaining substantially the same transcriptional factor activity.

As used herein, the percent identity between the two amino-acid sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino-acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4.

### Conditions for Increasing the Expression of T Box Transcription Factor

Any conditions available in the art for increasing expression of a T box transcription factor can be used in the methods of the invention, as long as such conditions result in the presence of T box transcription factor in a higher amount than what is normally observed in the original cells.

Various methods for increasing expression of reprogramming factors have been described in the art. For example, see “Pluripotency and cellular reprogramming: facts, hypotheses, unresolved issues”. Hanna J H, Saha K, Jaenisch R. Cell. 2010 Nov. 12; 143(4):508-25. Review.

In preferred embodiments, the following alternative may be used for increasing expression of a T box transcription factor:
(i) enhancing endogenous expression of the gene encoding said T Box transcription factor,
(ii) allowing ectopic expression of said T box transcription factor by introducing an expression vector comprising a coding sequence of T Box transcription factor operably linked to control sequences into the cells to be reprogrammed, or
(iii) introducing directly into the cells an appropriate amount of T Box transcription factor or its coding RNA.

In a first embodiment, enhancing endogenous expression of T Box transcription factor may be achieved for example either by
(i) modulating the signalling pathways controlling expression of T-Box factors in the PSM, including but not restricted to, FGF, BMP and Wnt signalling pathways,
(ii) introducing regulators of T-Box factors expression such as transcription factors, or
(iii) inhibiting the expression of inhibitors of T-Box factors by RNAi, shRNA, antisense oligonucleotides, dominant negative or chemical inhibitors.

For example, in a specific embodiment, endogenous expression of Brachyury may be enhanced by culturing the cells with an appropriate amount of enhancer factor(s), such as a protein activating the FGF signaling pathway, for example FGF8 or FGF4. Any other methods known in the art for stimulating, increasing or enhancing the expression of T Box transcription factor, for example, Brachyury, may be used in the method of the invention.

Thus, in a second embodiment, an expression vector comprising the T box transcription factor coding sequence, for example, Brachyury coding sequence, is introduced into the target cells. In one preferred embodiment, said Brachyury coding sequence comprises SEQ ID NO:3 (human Brachyury coding sequence) or SEQ ID NO:4 (mouse Brachyury coding sequence) or a coding sequence having at least 60%, 70%, 80%, 90% or 95% identity to SEQ ID NO:3 or SEQ ID NO:4.

The percent identity between two nucleotide sequences may be determined using for example algorithms such as the BLASTN program for nucleic acid sequences using as defaults a word length (W) of 11, an expectation (E) of 10, M=5, N=4, and a comparison of both strands.

Expression vectors for ectopic expression of the T Box transcription factors may be for example, plasmid vector, cosmid vector, bacterial artificial chromosome (BAC) vector, transposon-based vector or viral vector. In one specific embodiment, the expression vector used for increasing expression of T-box transcription factor is a viral vector. Examples of such viral vectors includes vectors originated from retroviruses such as HIV (Human Immunodeficiency Virus), MLV (Murine Leukemia Virus), ASLV (Avian Sarcoma/Leukosis Virus), SNV (Spleen Necrosis Virus), RSV (Rous Sarcoma Virus), MMTV (Mouse Mammary Tumor Virus), etc, Adeno-associated viruses, and Herpes Simplex Virus, but are not limited to.

Typically, the coding sequence of T Box transcription factor, for example, Brachyury, may be operably linked to control sequences, for example a promoter, capable of effecting the expression of the coding sequence in the cells to be reprogrammed. Such expression vector may further include regulatory elements controlling its expression, such as a promoter, an initiation codon, a stop codon, a polyadenylation signal and an enhancer. The promoter may be constitutive, or inducible. The vector may be self-replicable or may be integrated into the DNA of the host cell.

Alternatively, the vector for ectopic expression is a viral vector and viral particles are produced and used to introduce the coding sequence of said T Box transcription factor, for example, Brachyury, into said target cells. The term <<viral particles>> is intended to refer to the particles containing viral structural proteins and a sequence coding T Box transcription factor.

Viral particles may be prepared by transforming or transfecting a packaging cell with a viral vector carrying the nucleotide coding sequence of T Box transcription factor, for example, Brachyury. In the examples below, Brachyury-expressing viral particles are prepared from lentivirus. Viral particles can be used to infect the cells to be reprogrammed using transduction methods.

Incorporating the coding sequence and its control sequences directly into the genome of the target cells may cause activating or inactivating mutations of oncogenes or tumor suppressor genes, respectively. For certain applications, in particular medical applications, it may be required to avoid any genetic modifications of the target cells.

In a third embodiment, the T Box transcription factor, for example, Brachyury, or corresponding coding DNA or RNA, is introduced into the cells without integration of exogenous genetic material in the host DNA, i.e. without introduction of the nucleotide sequence in the cell's genome.

An expression vector such as a plasmid vector can be introduced into said cells for ectopic expression of T box transcription factor, in the form of naked DNA. Alternatively, RNA coding for Brachyury either chemically modified or not, can be introduced into the cells to reprogram them (see for example Warren L, et al, 2010).

These nucleic acids can be introduced with the aid, for example, of a liposome or a cationic polymer, for example, using conventional transfection protocols in mammalian cells.

Alternatively, the Brachyury protein or fragments thereof showing similar properties to the intact proteins with respect to the reprogrammation of iPSM can be introduced into said cells with the aid of chemical carriers such as cell-penetrating peptides such as penetratin or TAT-derived peptides.

### Conditions for Inhibiting at Least Retinoic Acid Signaling in Said Target Cells

In one preferred embodiment, said appropriate conditions for reprogramming said target cells into iPSM cells further comprise inhibiting at least retinoic acid signalling in said target cells.

Retinoic Acid (RA) is a small Vitamin A derivative, exhibiting pleiotropic effects during embryonic development. The signal transduction consists of direct binding of RA to the nuclear RA receptors and Retinoid X receptors (RARs and RXRs). These receptors act as ligand-dependent transcriptional activators of genes that contain RA-response elements (RAREs).

Any conditions may be used for inhibiting RA signalling in the method of the invention as long as these conditions result in significant and specific decrease of RA dependent transcription of RA-responsive genes.

In specific embodiments, said conditions for inhibiting retinoic acid signalling may be selected from the group consisting of:
a) allowing ectopic expression of a nucleic acid construct encoding a dominant negative retinoic acid receptor (dnRAR) in said target cells;
b) culturing the target cells in the presence of an appropriate amount of one or more compound inhibitors of retinoic acid receptor signalling or of the retinoic acid biosynthetic enzymes such as retinaldehyde dehydrogenase;
c) culturing the target cells in a medium depleted in retinoids;
d) inhibiting endogenous expression of one or more genes involved in retinoic acid signalling in said target cells; and,
e) overexpressing genes coding for retinoic acid inhibitors or involved in retinoic acid catabolism such as the Cyp26 enzyme.

Dominant negative retinoic acid receptor (dnRAR) have been described in the art (Damm et al., 1993). Ectopic expression of a gene encoding a dnRAR may therefore be achieved using similar expression vectors, such as non-viral or viral vectors as described in the above paragraph. For example, viral expression vectors comprising the gene encoding a dnRAR may be used. An example of coding sequence of dnRAR is the nucleotide sequence of SEQ ID NO:5, for use with human target cells. An example of coding sequence of dnRAR is the nucleotide sequence of SEQ ID NO:6 for use with mouse target cells.

Alternatively, any known compound inhibitors of retinoic acid signalling may be used as compound inhibitors of retinoic acid receptor. Such compound inhibitors may be selected from inhibitory nucleic acids, inhibiting the expression of retinoic acid receptor or a member of the retinoic acid receptor signalling pathway, for example, antisense oligonucleotides, siRNA, shRNA or miRNA. Such compound inhibitors may also be neutralizing or antagonist antibodies inhibiting or neutralizing one member of the retinoic acid receptor signalling pathway. Other compounds may be organic or inorganic molecules, small molecules, chemical or natural products known in the art to exhibit retinoic acid receptor antagonist properties. Examples of retinoic acid receptor antagonists include but are not limited to AGN 193109, AGN 190121, AGN 194574, AGN 193174, AGN 193639, AGN 193676, AGN 193644, SRI 11335, Ro 41-5253, Ro 40-6055, CD 2366, BMS493, BMS 185411, BMS 189453, CD 2665, CD 2019, CD 2781, CD 2665, CD 271.

Examples of retinaldehyde inhibitors include but are not limited to Disulfiram, and DEAB.

In one specific embodiment of the invention, the method for preparing iPSM cells comprises
(a) the direct introduction of the Brachyury transcription factor or corresponding coding RNA into said target cells in an amount sufficient for auto-induction of the endogenous expression of Brachyury transcription factor, and
(b) culturing the target cells in the presence of one or more inhibitors of retinoic acid signalling,
wherein said method does not involve any genetic modification of said target cells.

As used herein, the term “genetic modification”, refers to the stable introduction of a nucleic acid into the genome of a cell by artificial means.

Avoiding genetic modification of the cells is particularly advantageous for example in methods for preparing cells to be administered in human, for example, as a cell therapy product. The Brachyury transcription factor being auto-inducible (Conlon et al., 1996), the inventors have observed that introducing ectopic Brachyury in the cells is sufficient to activate endogenous expression of said Brachyury in said cells to be reprogrammed.

Furthermore, inhibiting retinoic acid signalling may be accomplished by incubating the cells in the presence of one or more compound inhibitors of retinoic acid signalling as hereabove described.

The invention also relates to a kit for preparing iPSM cells, said kit comprising
(a) means for increasing expression of T Box transcription factor, for example Brachyury, in a mammalian cell;
(b) means for inhibiting RA signalling in a mammalian cells; and,
(c) optionally, instructions for preparing iPSM cells.

In one specific embodiment, said kit for preparing iPSM cells comprises,
(a) a composition comprising Brachyury transcription factor or its corresponding coding RNA, and
(b) one or more compound inhibitors of retinoic acid signalling, for example, retinoic acid receptor antagonists.
   Compositions Comprising iPSM Cells Obtainable from the Methods of the Invention

The invention further relates to a composition comprising iPSM cells obtainable from the method as described above.

These compositions typically may comprise other cell types in addition to iPSM cells. In one embodiment, the compositions of the invention are characterized in that they comprise at least 10%, 20%, 30%, 40%, 50%, 60%, 70%, 80% and preferably at least 90% of cells that exhibit high expression of at least one biomarker characteristic of presomitic mesoderm cells, for example Msgn1 gene product.

Other biomarkers characteristic of presomitic mesoderm cells include, without limitation, one or more of the following proteins: EphrinA1, EphrinB2, EPHA4, Notch1, FGFR1, PDGFRalpha, Sall1, Sall4, Tbx6, Dll1, Thrombospondin2, N-Cadherin, Papc, VEGFR, Lfng, Hes7, Ripply1/2 or Mesp2.

Any methods known in the art for measuring gene expression may be used, in particular, quantitative methods such as, real time quantitative PCR, or methods using gene reporter expression, said gene reporter comprising Msgn1 promoter as described in the Examples, or qualitative methods such as immunostaining or cell sorting methods identifying cells exhibiting cell surface specific biomarkers.

As used herein, the Msgn1 gene refers to the gene encoding Mesogenin1. Examples of a nucleotide sequence of a gene encoding Mesogenin1 in mouse and human are given in SEQ ID NO:7 and SEQ ID NO:8 respectively.

In one embodiment, expression of Msgn1 is considered high if expression is detectable in a quantitative assay for gene expression. In another embodiment, it is high if the expression level is significantly higher than the expression level observed in the cultured cells to be reprogrammed under similar growth conditions. Expression levels between the control and the test cells may be normalized using constitutively expressed genes such as GAPDH.

Compositions comprising iPSM cells may be cultured indefinitely under appropriate growth conditions. Appropriate growth conditions may be established by the skilled person in the art based on established growth conditions for embryonic stem cells or induced pluripotent stem cells (iPS cells) for example or as described in the Examples below. Growth conditions may advantageously comprise for example the use of serum replacement medium, KSR, ESGRO supplemented with growth factors like FGFs, WNTs, BMPs or chemical compounds modulating the respective signalling pathways.

The iPSM cells may be purified or the compositions may be enriched in iPSM cells by selecting cells expressing markers specific of iPSM cells. In one embodiment, markers specific of iPSM cells for purification or enrichment of a composition of iPSM cells may be selected among one or more of the following markers Msgn1 gene product or EphrinA1, EphrinB2, EPHA4, Notch1, FGFR1, PDGFRalpha, Sall1, Sall4, Tbx6, Dll1, Thrombospondin2, N-Cadherin, Papc, VEGFR, Lfng, Hes7, Ripply1/2, Mesp2.

Purification or iPSM enrichment may be achieved using cell sorting technologies, such as FACS, or column affinity chromatography or magnetic beads comprising specific binders of said cell surface markers of iPSM cells.

After purification or enrichment, the composition may thus comprise more than 10%, 20%; 30%, 40%, 50%, 60%; 70%, 80%, 90% or more than 95% of cells having a high expression of a biomarker characteristic of iPSM cells, for example, Msgn1 gene product.

Methods for Preparing Cell Lineages by Differentiation of iPSM Cells

The iPSM cells may advantageously be cultured in vitro under differentiation conditions to generate muscle, cartilage, bone or dermal cells as well as other derivatives of the presomitic mesoderm including but not restricted to adipocytes or endothelial cells.

Thus, the invention relates to the methods for preparing compositions comprising muscle skeletal or dermal cell lineages, said method comprising the steps of
(a) providing a composition comprising iPSM cells; and,
(b) culturing said composition comprising iPSM cells, under appropriate conditions for their differentiation into the desired cell lineages selected among the presomitic mesoderm derivatives which include skeletal muscle, bone, cartilage or dermal cells.

The skilled person may adapt known protocols for differentiating stem cells, such as induced pluripotent stem cells, ES cells or mesenchymal stem cells into muscle, bone, cartilage or dermal cells.

In one specific embodiment, the present invention provides a method for preparing compositions comprising skeletal muscle cell lineages, said method comprising the steps of
(a) providing a composition comprising iPSM cells;
(b) culturing said composition comprising iPSM cells in the presence of a differentiation medium comprising at least the following components:
   (i) an extracellular matrix material; and,
   (ii) compounds activating or inhibiting the signalling pathways known to control of the differentiation of said lineages which include but are not restricted to retinoic acid, BMP, Hedgehog, Notch, FGF, Wnt, myostatin, insulin, PDGF, MAPK, PI3K, DNA methylation, DNA acetylation; and,
(c) optionally, culturing said composition obtained from step (b) in a second differentiation medium comprising at least one or more of the following differentiation factors bFGF, HGF, horse serum, Activin A, transferrin, EGF, insulin, LiCl, and IGF-1,
thereby obtaining a composition comprising skeletal muscle cell lineages.

The use of engineered extracellular matrices or three dimensional scaffolds has been widely described in the Art (Metallo et al., 2007). In specific embodiments, the extracellular matrix material is selected from the group consisting of Collagen I, Collagen IV, Fibronectin, gelatine, poly-lysine and Matrigel.

Several examples of suitable conditions for differentiating iPSM cells into skeletal or muscle cell lineages are described in Example 3 or 5 below.

In another embodiment, the present invention provides a method for preparing a composition comprising dermal cell lineages, said method comprising the steps of culturing a composition comprising iPSM cells in the presence of an efficient amount of at least one or more factors selected from the group consisting of BMP, Wnt, FGF, EGF, retinoic acid, and Hedgehog families of growth factors.

Examples of suitable conditions for differentiating iPSM cells in dermal cell lineages are described in Example 3 below.

In another specific embodiment, the present invention provides a method for preparing a composition comprising bone or cartilage cell lineages, comprising the step of culturing a composition comprising iPSM cells in the presence of an efficient amount of at least one or more factors selected from the group consisting of retinoic acid, Wnt, Hedgehog, pTHRP, TGF, BMP families of growth factors, dexamethasone, ascorbic acid, vitamin D3, and b-glycerophosphate.

Examples of suitable conditions for differentiating iPSM cells into bone or cartilage cell lineages are described in Examples 3 and 6 below.

In yet another embodiment, the present invention provides a method for preparing a composition comprising adipocyte derivatives, said method comprising the steps of culturing the composition comprising iPSM cells in the presence of an efficient amount of at least one or more factors selected from the group consisting of dexamethasone, isobutylxanthine and insulin.

Composition of Cells Derived from iPSM Cells and Uses Thereof.

Another aspect of the invention relates to the use of said composition comprising iPSM cells, or said composition comprising muscle, bone, cartilage or dermal cell lineages derived from differentiation of iPSM cells, hereafter referred as the Compositions of the Invention.

The Compositions of the Invention may be used in a variety of application, in particular, in research or therapeutic field.

One major field of application is cell therapy or regenerative medicine. For example, primary cells, such as fibroblast cells obtained from a patient suffering from a genetic defect may be cultured and genetically corrected according to methods known in the art, and subsequently reprogrammed into iPSM cells and differentiated into the suitable cell lineages for re-administration into the patient.

Similarly, regenerative medicine can be used to potentially cure any disease that results from malfunctioning, damaged or failing tissue by either regenerating the damaged tissues in vivo by direct in vivo implanting of a composition comprising iPSM cells or their derivatives comprising appropriate progenitors or cell lineages.

Therefore, in one aspect, the invention relates to the iPSM cells or their derivatives or the Compositions of the Invention for use as a cell therapy product for implanting into mammal, for example human patient.

In one specific embodiment, the invention relates to a pharmaceutical composition comprising iPSM cells, including for example at least 10², 10³, 10⁴, 10⁵, 10⁶, 10⁷, 10⁸, or at least 10⁹ Msgn1 expressing cells, and a pharmaceutically acceptable vehicle.

In one specific embodiment, the Composition of the Invention is used for the treatment of a muscle genetic disorder, for example Duchenne muscular dystrophy, or any other genetic muscular dystrophy.

In an embodiment, iPSM cells are co-cultured with various cell types to induce their differentiation toward the desired lineage. In another embodiment, iPSM cells are directly grafted into a recipient host. For regenerative medicine purposes, iPSM cells can be grafted after genetic corrections by methods known in the art.

In another specific embodiment, the Composition of the Invention is used for the treatment of joint or cartilage or bone damages in orthopaedic surgery caused by aging, disease, or by physical stress such as occurs through injury or repetitive strain.

In another specific embodiment, the Composition of the Invention may also be used advantageously for the production of dermal tissues, for example, skin tissues, for use in regenerative medicine or in research, in particular in the cosmetic industry or for treatment of burns.

In another specific embodiment, the Composition of the Invention may also be used advantageously for the production of but not restricted to dermal, muscle or skeletal cells from healthy or diseased patients for screening applications in the pharmaceutical industry. Such screening tests can be used to search for new drugs with clinical applications or for toxicology tests.

The invention will be further illustrated by the following figures and examples. However, these examples and figures should not be interpreted in any way as limiting the scope of the present invention.

### BRIEF DESCRIPTION OF THE FIGURES

FIG. 1 PSM markers Msgn1, Tbx6 and T are upregulated in iPSM cells (Venus positive cells) compared to the Venus negative cell population. Gene expression was quantified by quantitative real-time PCR and a TaqMan probe specific for the 3′UTR of endogenous T was used to avoid the detection of ectopically expressed T (lentiviral). All expression values of Venus positive cells (iPSM cells or MsgnRepV positive cells) (right) are normalized to expression values of the respective genes in Venus negative cells (left; values set to 1).

FIG. 2 (A,B) Response of iPSM cells to factors (Fgf8, Wnt3a, RA, Bmp4 and Shh) in the presence or absence of T-ires-dnRAR (doxycycline treated, Dox). For each experimental condition, the percentage of MsgnRepV positive cells (iPSM cells) was measured by FACS analysis and values are expressed as the ratio of treated iPSM cells relative to iPSM cells grown in absence of the factors without (A) or with doxycycline (B). (C-D) Time-course of primary cells microdissected from MsgnRepV E9.5 embryos (C) and reprogrammed iPSM cells (D) 0.5 to 3.5 days post sorting. Cells were grown in the presence of Wnt3a and Doxycycline. (Top: YFP channel, Bottom: bright field, magnification 20×). Representative FACS profiles for each condition (primary cells microdissected from MsgnRepV E9.5 embryos and reprogrammed iPSM cells) are shown on the left. Venus positive cells were separated from auto-fluorescent cells by comparing the Venus/GFP channel to a blue (PerCP) channel and only GFP single-positive cells were sorted.

FIG. 3 Myogenic differentiation of iPSM cells. MyoD, Myogenin (Myog) and Pax3 expression was measured by real-time PCR in iPSM cells cultured in myogenic differentiation medium compared to primary fibroblasts and undifferentiated iPSM cells (iPSM cells that were not cultured in the myogenic differentiation medium). Results are normalized to GAPDH (N.D.: not detected).

FIG. 4 Chondrogenic differentiation of iPSM cells. Sox9 and Col2A1 expression was measured by real-time PCR in iPSM cells cultured in chondrogenic differentiation medium compared to primary fibroblasts and undifferentiated iPSM cells (iPSM cells that were not cultured in the chondrogenic differentiation medium). Results are normalized to GAPDH (N.D.: not detected).

### EXAMPLES

### Methods

### Cloning of Lentiviral Vectors:

To establish an inducible lentiviral expression system, the Lenti-X Tet-Off Advanced Inducible Expression System from Clontech was used. The system consists of two different vectors, i.e. the pLVX-Tet-Off Advanced vector, which is used to produce lentivirus expressing the tetracycline controlled transcriptional activator tTA, and the pLVX-Tight-Puro vector, into which the gene of interest is being cloned downstream of a tetracycline-responsive promoter. A co-infection with lentivirus generated with both vectors facilitates the generation of cells expressing the gene of interest under the control (repressible by) of tetracycline (or doxycycline). For all pLVX-Tight-Puro-based overexpressions, the coding sequence of the gene of interest is PCR-amplified, thereby introducing a Kozak sequence around the start codon (GCCACCATG), and inserted into the NotI-EcoRI sites of the multiple cloning site. For the constructs expressing the constitutively active form of Mkk1 (caMkk1) (Mansour et al., 1994), the stabilized form of b-catenin (dBC) (Harada et al., 1999) and the dominant negative retinoic acid receptor (dnRAR403) (Damm et al., 1993), the respective coding sequences are analogously amplified and initially cloned into pENTR-D-TOPO using the pENTR-D-TOPO cloning kit (Invitrogen), and successively recombined using the Invitrogen Gateway System (Gateway BP Clonase II enzyme mix, Invitrogen,) into a modified pLVX-Tight-Puro, which contains a Gateway cassette cloned between the NotI-EcoRI sites (using the Reading Frame Cassette A of the Gateway Vector Conversion System, Invitrogen).

### Production of Lentivirus:

Lentiviruses were produced in HEK293T cells using the Lenti-X HT Packaging System (Clontech) or the Lenti-X HTX Packaging System (Clontech), followed by ultracentrifugation.

### Medium for Cell Culture:

The medium used for the preparation of primary fibroblasts, infection, culture for reprogramming, maintenance/amplification and differentiation (unless otherwise stated) is DMEM (HIGH GLUCOSE w/o L-GLUTAMINE containing non-essential amino acids, Invitrogen, supplemented with GLUTAMAX, Invitrogen, Sodium Pyruvate, Invitrogen, and Penicilline [10.000 U/ml]/Streptomycin [10 mg/ml] Invitrogen) containing 10% Tet System Approved Fetal Bovine Serum (Clontech).

### Preparation of Mouse Fibroblasts:

Mouse fetuses of CD1 females (Charles River) mated to MsgnRepV homozygous reporter males are harvested 15.5 days post coitum (dpc). After removal of heads and liver, the fetuses are pressed through a syringe (without needle) and washed (@1000 rpm) once using 1×PBS (Clontech, Dulbecco, without Mg2+ and Ca2+, Invitrogen). The cell clumps are next digested using a 10:1 mixture of Collagenase IV (10 mg/ml, Invitrogen, reconstituted in PBS with Mg2+ and Ca2+, Invitrogen) and Dispase (50 U/ml, Invitrogen, reconstituted in 1×PBS without Mg2+/Ca2+) for 20 minutes at 37 C, with gentle shaking. Next, 2 volumes (relative to Dispase) of TryplE Express (Invitrogen) are added and the suspension is incubated for another 10 minutes (37 C, with agitation). After digestion, the suspension is washed (@1000 rpm) three times with Geneticin (Clontech)—containing Medium, and cells are plated at different dilutions into multiple wells of 6-well plates to obtain primary fibroblasts at a confluency of approximately 50% on the next day.

### Lentiviral Infection of Cells:

The day after preparation, the primary fibroblasts are infected with the lentiviral cocktail (in 6-well plates; at 50-80% confluence) using the ViraDuctin™ Lentivirus Transduction Kit (Cell Biolabs, Inc., San Diego, Calif., USA) in a volume of 1-2 ml per well (of the E-well plate) in Geneticin-containing medium. One day after primary fibroblast preparation, the medium is removed, and a second lentiviral infection with the same lentiviral cocktail is performed under identical conditions to day 1. On day 3, the medium is replaced with 2 ml of Geneticin-containing medium and cultured for another two days. From day 5 on, medium is replaced every 2-3 days (or more often for later, dense cultures, if medium turns yellow) with medium containing the appropriate selection.

### Flow Cytometry (FACS):

For analysis or sorting using flow cytometry, cells are trypsinized (using TryplE) until the majority of the dish contains single cells, mixed with 4 volumes of medium to inhibit TryplE, and washed 2 times with 1×PBS (@1000 rpm). After the 2nd wash, cells are resuspended in a small volume (less or equal 1*10̂6 cells) of PBS (for analysis) or 1% Tet System Approved Fetal Bovine Serum in PBS (for sorting). Clumps of multiple cells are removed using a 70 um Filcon (BD Biosciences). Venus positive cells (iPSM cells) are separated from auto-fluorescent cells by comparing the Venus channel to EGFP or Cerulean. If sorting is performed, cells are collected in medium during the sorting procedure and then plated at high density (minimum of 100.000 cells per well of a 48-well plate), since culturing of sorted cells at low densities leads to cell death.

### Differentiation:

For differentiation, iPSM cells are FACS sorted and plated in plates coated with different extracellular matrices. For a typical experiment, approximately 100.000 cells are plated per well in a 48-well plate that was previously coated with 100 ul of Matrigel (MATRIGEL PHENOL-RED free 40234C, BD Biosciences). After attachment of the cells, the medium is replaced on the same day with differentiation medium containing 10-100 ng/ml Doxycycline (Doxycycline Hydrochloride 98%, Sigma) with and without additional factors. For differentiation towards skeletal lineage, the medium is supplemented with 200 ng/ml Bmp4 (recombinant mouse Bmp4, R&D Systems), and for differentiation towards the muscle and dermis lineage, medium containing retinoic acid (1 uM/ml, ALL trans Retinoic Acid, 85%, Sigma) and 10 uM LiCl (Sigma) is used. For the differentiation into myotubes, the Retinoic Acid/LiCl containing medium is replaced with medium containing 2 ng/ml IGF-1 (Insulin-like Growth Factor 1, R&D Systems), 10 ng/ml HGF (recombinant mouse Hepatocyte Growth Factor, R&D Systems) and 2 ng/ml bFGF (basic Fibroblast Growth Factor, R&D Systems).

### Immunohistochemistry:

After culture, cells were fixed using 4% paraformaldehyde in PBS for 10 minutes, permeabilized in 0.2% Triton-X and blocked in either 0.1% gelatine or 10% goat serum. Antibody staining was performed over night at 4° C., followed by incubation with the fluorescent labelled secondary antibody for 1 hour at room temperature.

### Results

### Initial Screen for Transcription Factors

We initially focused on candidate transcription factors for which functional studies suggest a crucial involvement in the formation of the PSM or whose gene expression was largely confined to the PSM. The candidates were initially evaluated using chicken embryo electroporation for their ability to promote overexpressing cells to

(a) essentially contribute to the PSM (good candidates are expected to only ingress into the PSM, but not neural tube and/or intermediate/lateral plate mesoderm),
(b) shift anteriorly the expression of PSM markers Msgn1 and Tbx6 indicating a maintenance of the immature posterior PSM fate and
(c) prevent overexpressing cells to become incorporated into somites.

The candidate transcription factors were cloned in a plasmid downstream of the CAGGS promoter driving ubiquitous expression, and electroporated into the PSM progenitor cells in the primitive streak of an early chicken embryo. Of all candidates tested (n=53 genes selected from 120 genes with specific expression pattern after an in situ hybridization screen), only Msgn1-, Tbx6- and T-electroporated cells exclusively migrated into the PSM, and failed to down-regulate expression of Tbx6 and to form somites.

### Lentiviral Infection

We then tested the ability of these transcription factors to reprogram mouse somatic cells to a posterior PSM fate. To this end, we generated lentiviral vectors to force expression of the three transcription factors alone or in combination. In order to follow more precisely the reprogrammation of somatic cells toward the posterior PSM fate, we generated a transgenic mouse line (MsgnRepV) harboring a paraxial mesoderm-specific fluorescent reporter. In this mouse, expression of Venus (a modified yellow fluorescent protein YFP) is driven by the promoter of the mouse Msgn1 gene, which is specific to the undifferentiated posterior PSM (Yoon et al. 2000; Yoon and Wold 2000; Wittler et al. 2007). In these embryos, Venus mRNA expression is restricted to the endogenous Msgn1 expression territory in the posterior PSM. Embryos obtained from this mouse line exhibits fluorescently labeled PSM tissue.

We then generated primary fibroblasts from E15.5 MsgnRepV mouse embryos and subjected them to infection with combinations of the various lentiviral constructs expressing Msgn1, Tbx6 (Agulnik et al. 1996; Chapman et al. 1996a; Chapman et al. 1996b; Hug et al. 1997; Knezevic et al. 1997; Chapman and Papaioannou 1998) and T (Wilkinson et al. 1990; Kispert and Hermann 1993; Rashbass et al. 1994; Yamaguchi et al. 1999; Gadue et al. 2006). Remarkably, cells overexpressing combinations of factors containing the T gene cultured for 4 weeks, showed up to 2.5% fluorescent cells.

The posterior PSM in mouse and chicken is characterized by high levels of FGF, and WNT signaling and low levels of retinoic acid (RA) signaling. We generated lentiviral constructs constitutively activating FGF (constitutive-active Map kinase, caMkk1, leading to high levels of phospho-ERK), WNT (non-degradable beta-catenin, dBC, thereby keeping canonical WNT/beta-catenin signaling in active state), and a dominant-negative Retinoic Acid Receptor A (dnRAR, inhibiting RA targets), respectively. We next subjected MsgnRepV fibroblast to infection with combinations of the various lentiviral constructs expressing Msgn1, Tbx6 and T as well as with the constructs triggering constitutive-active beta-catenin signaling (dBC), constitutive-active Map kinase signaling (caMkk1) and dominant-negative retinoic acid signaling (dnRAR). The lentiviral infection was performed using the Lenti-X tetracycline repressible system from Clontech, with modifications. The 6 constructs were used alone or in combinations as listed in Table 1, leading to a total of 24 infections per experiment.

While no positive cells were observed by flow cytometry (FACS) after 2 weeks in the initial experiments, for some of the combinations, a population of Msgn1 positive cells separated during FACS analysis. The experiment was then repeated another 3 times (two experiments were carried out with E9.5 and E10.5 fibroblasts respectively) and, as summarized in table 1, several combinations containing T gave rise to Msgn1 positive cells, with the highest rate for the combination of T and dnRAR.

TABLE 1

| | Stage, duration of infection | | | | | |
|---|---|---|---|---|---|---|
| | E10.5 | E15.5 | E15.5 | E9.5 | E15.5 | E15.5 |
| Factors | 5 weeks¹ | 4 weeks² | 6 weeks³ | 4 weeks⁴ | 4 weeks⁵ | 6 weeks⁶ |
| Msgn1 + caMkk1 | 0.00 | 0.00 | 0.00 | 0.01 | 0.00 | 0.01 |
| Tbx6 + caMkk1 | 0.00 | 0.00 | 0.00 | 0.00 | 0.00 | 0.00 |
| T + caMkk1 | 0.00 | 0.34 | 0.21 | 0.00 | 0.11 | 0.57 |
| Msgn1 + Tbx6 + T + | 0.00 | 0.04 | 0.01 | 0.00 | 0.00 | 0.00 |
| caMkk1 | | | | | | |
| Msgn1 + dBC | 0.01 | 0.03 | 0.00 | 0.02 | 0.00 | 0.01 |
| Tbx6 + dBC | 0.01 | 0.00 | 0.00 | 0.00 | 0.00 | 0.00 |
| T + dBC | 0.00 | 0.73 | 0.25 | 0.01 | 0.00 | 0.22 |
| Msgn1 + Tbx6 + T + dBC | 0.00 | 0.07 | 0.06 | 0.01 | 0.00 | 0.00 |
| Msgn1 + dnRAR | 0.01 | 0.00 | 0.00 | 0.01 | 0.00 | 0.01 |
| Tbx6 + dnRAR | 0.00 | 0.00 | 0.00 | 0.00 | 0.00 | 0.00 |
| T + dnRAR | 1.07 | 4.09 | 9.86 | 0.90 | 1.06 | 3.30 |
| Msgn1 + Tbx6 + T + | 0.00 | 0.19 | 0.00 | 0.01 | 0.01 | 0.00 |
| dnRAR | | | | | | |
| Msgn1 + caMkk1 + dBC + | 0.00 | 0.00 | 0.00 | 0.00 | 0.00 | 0.00 |
| dnRAR | | | | | | |
| Tbx6 + caMkk1 + dBC + | 0.00 | 0.00 | 0.00 | 0.00 | 0.00 | 0.01 |
| dnRAR | | | | | | |
| T + caMkk1 + dBC + | 0.00 | 1.01 | 0.00 | 0.00 | 0.09 | 0.09 |
| dnRAR | | | | | | |
| Msgn1 + Tbx6 + T + | 0.00 | 0.00 | 0.00 | 0.00 | 0.00 | 0.00 |
| caMkk1 + dBC + dnRAR | | | | | | |
| Msgn1 | 0.01 | 0.00 | 0.00 | 0.00 | 0.00 | 0.00 |
| Tbx6 | 0.00 | 0.00 | 0.00 | 0.01 | 0.00 | 0.00 |
| T | 0.28 | 1.55 | 2.50 | 0.01 | 0.89 | 1.92 |
| Msgn1 + Tbx6 + T | 0.00 | 0.13 | 0.24 | 0.01 | 0.01 | 0.00 |
| caMkk1 | 0.00 | 0.01 | 0.01 | 0.02 | 0.00 | 0.01 |
| dBC | 0.00 | 0.00 | 0.00 | 0.00 | 0.00 | 0.01 |
| dnRAR | 0.00 | 0.01 | 0.00 | 0.00 | 0.00 | 0.03 |
| caMkk1 + dBC + dnRAR | 0.01 | 0.00 | 0.00 | 0.00 | 0.00 | 0.00 |

Percentage of Msgn-Venus positive cells (based upon FACS analysis) in 4 independent experiments for virus combination indicated in first column.
³Same sample as², but re-cultured for additional 2 weeks after analysis.
³cultured for 1 week before infection.
⁶Same sample as⁵, but re-cultured for additional 2 weeks after analysis.

Since the initial experiment did not contain all possible combinations of the 6 factors, we designed a second set of experiments, this time combining the remaining factors (Msgn1, Tbx6, caMkk1, dBC) with T and dnRAR (Table 2). However, none of these mixtures increased the percentage of positive cells over the T+dnRAR reference (Table 2).

TABLE 2

| | Stage, duration of infection | | | |
|---|---|---|---|---|
| | E15.5 | E15.5 | E15.5 | E15.5 |
| Factors | 4 weeks¹ | 4 weeks² | 6 weeks³ | 6 weeks⁴ |
| Msgn1 + T + dnRAR | 0.00 | 0.02 | 0.06 | 0.00 |
| Tbx6 + T + dnRAR | 0.10 | 0.54 | 0.70 | 0.28 |
| Msgn1 + caMkk1 + T + | 0.09 | 0.03 | 0.00 | 0.02 |
| dnRAR | | | | |
| Tbx6 + caMkk1 + T + | 0.37 | 0.09 | 0.09 | 0.32 |
| dnRAR | | | | |
| Msgn1 + dBC + T + | 0.00 | 0.00 | 0.00 | 0.01 |
| dnRAR | | | | |
| Tbx6 + dBc + T + | 0.03 | 0.43 | 0.85 | 1.46 |
| dnRAR | | | | |
| caMkk1 + T + dnRAR | 0.07 | 0.31 | 0.44 | 0.11 |
| dBC + T + dnRAR | 0.37 | 0.12 | 0.47 | 2.01 |
| T + dnRAR | 1.14 | 3.36 | 1.69 | 4.44 |
| T + dnRAR | 0.96 | 1.10 | 2.94 | 3.82 |

Percentage of Msgn1-Venus positive cells (based upon FACS analysis) in 2 independent experiments for virus combination indicated in first column.
³Same sample as¹, but re-cultured for additional 2 weeks after analysis.
⁴Same sample as², but re-cultured for additional 2 weeks after analysis.

Strikingly, the T+dnRAR expressing cells exhibited long term self renewing properties, since it was possible to maintain them in culture indefinitely. A population of more than 10% of Msgn1 positive iPSM cells was maintained in culture for more than 10 months. Infection of newborn dermal skin fibroblasts with the same two lentiviruses gave essentially similar results. We then constructed a lentiviral vector to express a bicistronic construct driving expression of T and dnRAR linked by an IRES (Internal Ribosomal Entry Site). This construct was used to infect both E15.5 embryos and newborn dermal skin fibroblasts which showed between 5 to 10% venus positive cells after 4 weeks of culture.

### Characterization Of Msgn1 Positive Cells

We used FACS analysis to efficiently sort Msgn1-positive cells from infected fibroblast cultures. The sorted positive cells were subjected to quantitative real-time PCR using TaqMan probes specific for the PSM markers Msgn1 and Tbx6, as well as for a 3′ UTR specific custom probe for T (which only detects endogenously expressed T), and compared to sorted negative cells and to non-infected fibroblasts. The expression of the three PSM markers was significantly upregulated when comparing positive and negative FACS-sorted cells. In contrast, the expression levels of differentiation markers for the respective somitic lineages were significantly below the expression level detected in the somites of mouse embryos.

### Differentiation

We next examined the ability of the Msgn1 positive cells infected with the T and dnRAR expressing viruses to differentiate into normal presomitic mesoderm derivatives, ie muscle, dermis and skeletal lineages. Using FACS, we sorted Msgn1 positive cells from long-term cultures (10 months) and explored their ability to differentiate into muscle or skeletal lineages. Based upon initial experiments with different matrix-coated plates (Collagen I, Collagen IV, Fibronectin, Matrigel), and a combination of different proteins/chemicals activating signaling pathways required for the respective differentiation in vivo (Bmp4, RA, LiCl, Shh; alone and in combination), we developed the following differentiation protocol: After being sorted by Flow cytometry, cells were re-plated in wells in tetracycline containing medium in a 48-well plate coated with 100 ul of Matrigel. The cultured cells were then treated for 5 and 10 days with either Bmp4 or a combination of RA and LiCl. After induction with Bmp4 and RA+LiCL, the expression of the sclerotomal/chondrocyte/osteoblasts markers Col2a1, Sox9 and Pax1 and the myotomal/myocyte markers Myf5, Myod and Pax3 respectively was elevated after 5 and 10 days. The dermis markers En2 and Dermo1 also were upregulated using both combinations. Msgn1-positive cells, were allowed to differentiate on Matrigel for 2-3 weeks, and then fixed and processed for immuno-staining using different muscle-specific antibodies. Polynucleated cells exhibiting a myofiber-like morphology, which tested positive against MF20 (muscle sarcomeres), HHF35 (muscle actin), A4.1025 (myosin, all fibers) and F1.652 (myosin, embryonic) were present in cultures differentiated for 3-6 days with RA+LiCl, followed by differentiation medium containing bFGF, HGF and IGF-1.

### Example 1

### Method for Preparing a Composition Comprising iPSM Cells from Primary Fibroblasts

Reprogrammation experiments of human primary fibroblasts to iPSM cells are performed using low-passage primary fibroblasts acquired from commercial vendors or human biopsies.

In the case of skin biopsies, the samples are first washed in 1×PBS (Clontech, Dulbecco, without Mg2+ and Ca2+, Invitrogen). Next, the skin is exposed to a 10:1 mixture of Collagenase IV (10 mg/ml, Invitrogen, reconstituted in PBS with Mg2+ and Ca2+, Invitrogen) and Dispase (50 U/ml, Invitrogen, reconstituted in 1×PBS without Mg2+/Ca2+) for 20 minutes at 37 C, with gentle shaking, followed by an additional 10 minutes of incubation (37 C, with agitation) after addition of 2 volumes (relative to Dispase) of TryplE Express (Invitrogen). Next, the cells are collected using centrifugation (@1000 rpm) and washed 3 times in cell culture medium before being plated in 6-well plates, aiming 50% at confluency after overnight incubation. On the next day, the freshly established (or freshly recovered, in the case of commercial vendors) fibroblasts are infected with a mixture of lentivirus comprising of the tet-off system together with virus particles derived from pLVX-tight driven human T and dnRAR. Optionally, a fluorescent reporter is co-introduced into the cells driven by the human promoter for MSGN1. The infected cells are then cultured under appropriate conditions until the presence of iPSM cells is detected by either fluorescence (in case of a fluorescent reporter being used) or by other methods like quantitative real-time RT-PCR or immunohistochemistry with PSM-specific markers.

Several different cell culture media may be used for the iPSM reprogramming experiments, including the cell culture medium mentioned above or specialized media established for the culture of human dermal fibroblasts like Medium 106 (Invitrogen) supplemented with Low Serum Growth Supplement (Invitrogen). Supplements can also be added to the culture medium including recombinant human or mouse growth factors of the BMP, FGF, or WNT families or compounds modulating the activities of these growth factors.

### Example 2

### Method for Growing and/or Sorting iPSM Cells

Using similar culture conditions as for the mouse counterparts, human iPSM cells derived from commercial fibroblasts or from tissue biopsies are cultured and propagated until a sufficient percentage of the initial cultures are reprogrammed into MSGN1/TBX6 positive cells. The percentage of iPSM cells can be assessed by either FACS (in case fluorescent reporters are used) or by quantitative real-time RT-PCR using PSM-specific markers such as MSGN1 or TBX-6. FACS sorting using several cell surface proteins specific to the PSM like EPHA1, DLL1, Thrombospondin2, N-Cadherin or PDGFR-alpha (alone or in combination) can be used to increase the percentage of MSGN1/TBX6 positive cells.

### Example 3

### Method for Inducing Differentiation into Muscle, Dermal or Skeletal Cell Lineages

iPSM cultures with a high percentage of positive cells (achieved through either optimized culture conditions or FACS sorting from iPSM cells obtained as described in Examples 1 and 2) are cultured on cell culture dishes for 4 days on coated plates with appropriate extracellular matrix extract such as Collagen IV in SF-03 medium containing 5 mM LiCl, followed by a re-plating on Collagen I coated plates and then cultured for 3-4 days in SF-03 medium supplemented with bFGF, HGF and IGF-1, and another 4 days in SF-03 IGF-1 containing medium in order to obtain Myogenin positive myofibers.

Alternatively, iPSM cells can be differentiated in two-dimensional culture into muscle cells using SF03 medium complemented with BMP4, ActivinA and IGF-1 for 3 days, followed by 3 days of SF03 medium complemented with LiCl and Shh.

iPSM cells can be cultured by hanging drop for 3 days at 800 cells/20 uL in differentiation medium, composed of DMEM (DMEM) supplemented with 10% fetal calf serum (FCS), 5% horse serum (Sigma), 0.1 mM 2-mercaptoethanol, 0.1 mM nonessential aminoacid, and 50 ug/ml penicillin/streptomycin. After 3 days, the medium is changed and cell aggregates are transferred on low attachment plate. At day 6, cells are plated and cultured in differentiation medium on plates coated with Matrigel (BD Bioscience, Bedford, Mass., USA). Myogenic differentiation is achieved by withdrawal of FBS from confluent cells and addition of 10 ug/ml insulin, 5 ug/ml transferrin, and 2% horse serum.

iPSM cells can also be cultured for 3 weeks in Skeletal Muscle Cell Medium (Lonza) complemented with EGF, insulin, Fetuin, dexamethasone, and bFGF (100 ng/mL).

For skeletal lineages iPSM cells are exposed to 200 ng/ul human or mouse recombinant BMP4 or a combination of 1 uM retinoic acid and 10 mM Lithium Chloride. Alternatively, cells are plated on gelatin-coated plates at a density of 1-3×10̂3 per well (24-well plate) and cultured for 28 days in bone differentiation medium (DMEM, 10% FBS, 2 mM 1-Glutamine, 1× Penicilin/streptomycin (P/S), 0.1 μM dexamethasone, 50 μM ascorbic acid 2-phosphate, 10 mM β-glycerophosphate, 10 ng/mL BMP4) in order to observe cells expressing bone specific markers or secreting alcian blue positive extracellular matrix. Differentiated skeletal cell lineages are identified using specific stainings for extracellular matrix components of bone and cartilage including alcian blue or alizarin red, as well as by immunofluorescence using chondrocyte- and/or osteocyte specific antibodies.

iPSM cells can also be differentiated into the bone lineage using the following differentiation medium composed of DMEM, 10% FBS, 2 mM L-Glutamine, 1×P/S, 0.1 mM Dexamethasone, 50 mM ascorbic acid 2-phosphate, 10 mM b-glycerophosphate, and 10 ng/mL BMP4, and vitamin D3 for 20 days, medium changed every 3 days. Bone formation can be confirmed by Alizarin red staining of the differentiating culture, well known in the art, that results in the staining of differentiated bone in red color. Extracellular accumulation of calcium can also be visualized by von Kossa staining. Alternatively, differentiating cells can be lysed and assayed for ALP activity using BBTP reagent. Alternatively, differentiating cells can be analyzed for osteoblast lineage markers expression, for example Osterix(Osx) and Cbfa1/Runx2, alkaline phosphatase, collagen type I, osteocalcin, and osteopontin.

For chondrogenic cell differentiation, iPSM cells are plated at a density of 8×10̂4 per well (24-well plate) and cultured for 30 minutes in a 37 C incubator in cartilage cell differentiation medium (αMEM, 10% FBS, 2 mM 1-Glutamine, 1×P/S, 0.1 μM Dexamethasone, 170 μM ascorbic acid 2-phosphate). Next, an equal amount of cartilage cell differentiation medium with 10 ng/mL TGF beta3 is added to the well. After one week, the medium is replaced with cartilage differentiation medium supplemented with 10 ng/mL Bmp2. After 21 days cartilaginous nodules secreting extracellular matrix can be observed. iPSM cells can also be differentiated into cartilage cells using a differentiation medium based on aMEM, 10% FBS, 2 mM L-Glutamine, 1×P/S, 0.1 mM Dexamethasone, and 170 mM ascorbic acid 2-phosphate or DMEM supplemented with 0.1 mM dexamethasone, 0.17 mM ascorbic acid, 1.0 mM sodium pyruvate, 0.35 mM L-proline, 1% insulin-transferrin sodium, 1.25 mg/ml bovine serum albumin, 5.33 ug/ml linoleic acid, and 0.01 ug/ml transforming growth factor-beta), as well as TGFb3 or BMP2. Cells are cultured for several weeks, with medium changed every 3 days. Differentiation can also be performed at high density on 3D scaffold such as Alginate beads in a DMEM based medium containing 10% FBS and antibiotic supplemented with 100 ng/ml recombinant human Bone Morphogenic Protein-2 (BMP-2) and 50 mg ascorbic acid. Cartilage formation can be confirmed by Alcian Blue staining of the differentiating culture, well known in the art, that results in the staining of Muco-glycoproteins in blue color. Alternatively, a safranin O staining can be performed.

iPSM cells can be differentiated into dermal fibroblasts by culturing them on a scaffold of collagen in medium containing a fibroblast growth factor such as bFGF (basic Fibroblast Growth Factor) or a member of the Wnt family of growth factors.

### Example 4

### Characterization of iPSM Cells

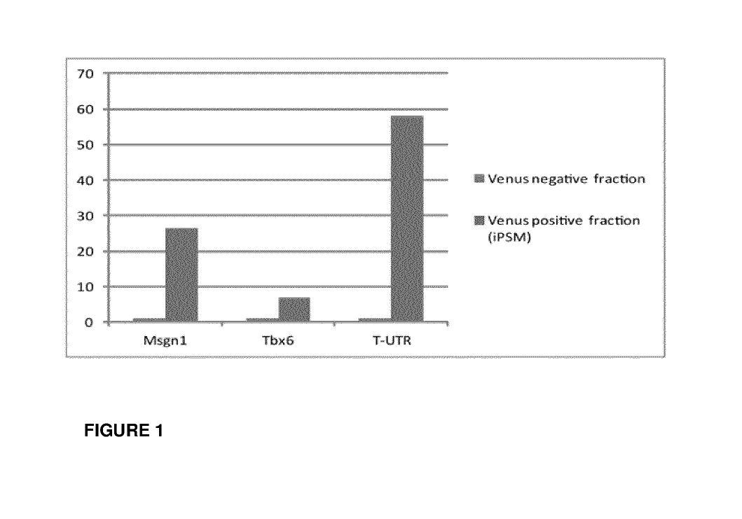

iPSM cells were successfully generated from embryonic and postnatal mouse dermal fibroblasts (Data not shown). After FACS sorting, positive cells can be re-plated and continuously grown on fibronectin-coated cell culture plates, although a certain percentage of positive cells turn off the MsgnRepV reporter after 1-2 weeks. When performing real-time PCR on the sorted cells, an upregulation of Msgn1, Tbx6 and endogenous T can be observed in MsgnRepV positive cells (iPSM cells) (using commercial TaqMan probes for Msgn1 and Tbx6, and a custom TaqMan probe for T which only detects endogenous transcript) (FIG. 1).

We supplemented the culture medium of iPSM cells with Fgf8 (R&D systems, Cat. No. 423-F8-025/CF), Wnt3a (R&D systems, Cat. No. 1324-WN-010/CF), retinoic acid (RA; Sigma Aldrich, Cat. No. R-2625), Bmp4 (R&D systems, Cat. No. 5020-BP-010) and Shh (R&D systems, Cat. No. 461-SH-025), both in the presence or absence (after addition of doxycycline) of the T-IRES2-dnRAR transcript.

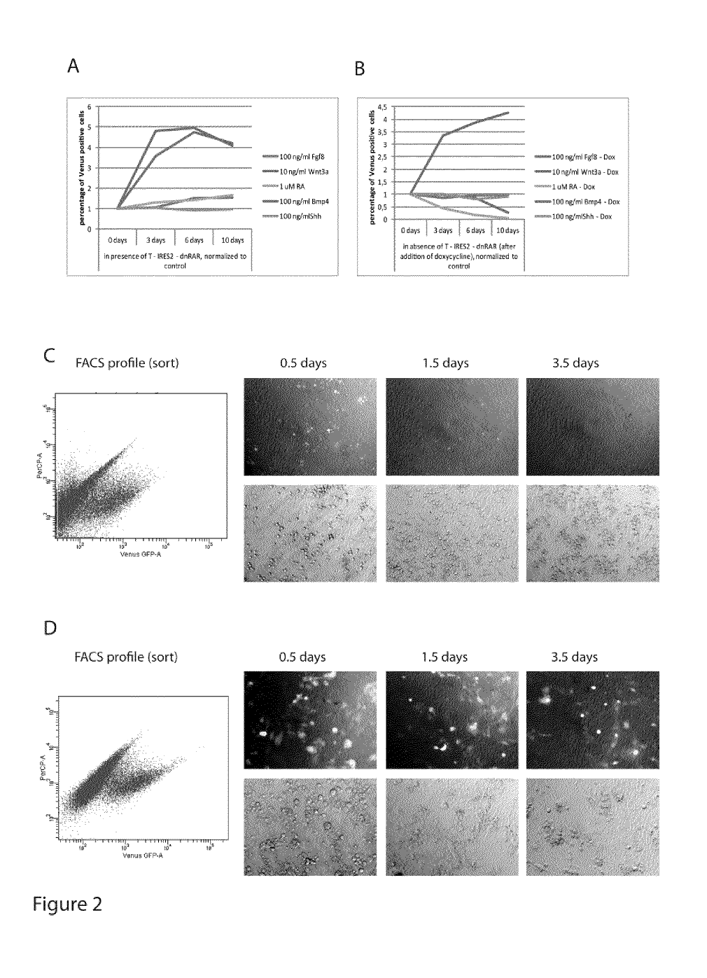

In the presence of the T-IRES2-dnRAR transcript, the percentage of iPSM (MsgnRepV positive cells) increases upon addition of Fgf8 and Wnt3a (FIG. 2A, see the two upper lines up to 4 at 10 days). In the absence of the T-IRES2-dnRAR transcript, Wnt3a is still capable of increasing the percentage of MsgnRepV positive cells (FIG. 2B, see the upper line (until 4.2 at 10 days)), while Fgf8 is no longer is able to increase the percentage of MsgnRepV positive cells. Additionally, in the presence of RA, cells rapidly lose the expression of MsgnRepV, suggesting that, like in the embryo, cells lose Msgn1 expression as they differentiate.

However, iPSM cells exhibit several properties that significantly distinguish them from endogenous embryonic PSM cells. Unlike mouse embryonic PSM cells, which in vivo undergo maturation within approximately half a day, iPSM cells show stem cell like characteristics since they can be maintained indefinitely. Even after addition of doxycycline, which leads to the downregulation of exogenous T and DN-RAR expression, iPSM cells remain positive for the MsgnRepV reporter.

We further investigated this difference and compared iPSM cells and primary PSM cells isolated from MsgnRepV transgenic embryos. After sorting the Venus positive fraction (YFP positive), cells were plated on fibronectin-coated dishes and cultured in Wnt3a and doxycycline containing medium, ensuring that, like in embryonic PSM cells, no transgene was ectopically expressed in iPSM cells (FIG. 2C-D). Our results indicate that primary embryonic PSM cells do not exhibit this stem-cell like properties, since they rapidly lose the reporter expression (within 2 days, see FIG. 2C), while reprogrammed iPSM cells maintain the MsgnRepV expression (FIG. 2D).

### Example 5

### Myogenic Differentiation

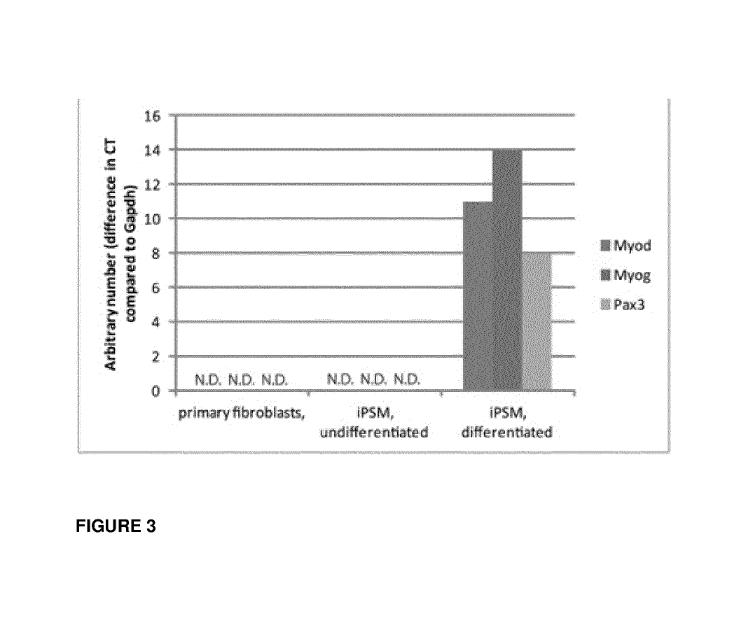

iPSM cells were FACS sorted and plated on fibronectin-coated 48-well tissue culture plates. Cells were grown in growth medium composed of DMEM HIGH GLUCOSE w/o L-GLUTAMINE (Invitrogen), containing non-essential amino acids (Invitrogen), GLUTAMAX (Invitrogen), Sodium Pyruvate (Invitrogen), Penicilline [10.000 U/ml]/Streptomycin [10 mg/ml] (Invitrogen), and 10% Tet System Approved Fetal Bovine Serum (Clontech). After 7 days, the growth medium was supplemented with 10 ng/ul Wnt3a in the presence of doxycycline. On day 14, when approximately 25% of the cells expressed the reporter at high levels, cells were split onto matrigel-coated 48-well tissue culture plates and cultured as follows:
1 day [day 15] in growth medium supplemented with 1 uM retinoic acid (RA; Sigma Aldrich, Cat. No. R-2625) and 10 uM 5-Azacytidine (Sigma Aldrich, Cat. No. A-1287).
1 day [day 16] in growth medium supplemented with 1 uM RA, 100 ng/ml HGF (R&D systems, Cat. No. 2207-HG-025/CF), 2 ng/ml bFGF (R&D systems, Cat. No. 3139-FB-025/CF), 2 ng/ml IGF1 (R&D systems, Cat. No. 791-MG-050), 10 ng/ml Wnt4 (R&D systems, Cat. No. 475WN-005), and 1 ug/ml Shh (R&D systems, Cat. No. 461-SH-025) or 200 nM SAG (smoothened antagonist, Calbiochem, Cat. No. 566660-1MG).
2 days [days 17-18] in growth medium supplemented with 1 uM RA, 100 ng/ml HGF, 2 ng/ml bFGF, 2 ng/ml IGF1, and 1 ug/ml Shh or 200 nM SAG.
   Next, RNA was extracted using Trizol (Invitrogen, Cat. No. 15596-026), and one-step quantitative real-time PCR (QuantiFast Multiplex RT-PCR Kit, Cat. No. 204854) was performed using a Roche LightCycler II and TaqMan probes for Myod (Applied Biosystems, Cat. No. Mm00440387_m1), Myogenenin (Myog) (Cat. No. Mm00446195_g1) and Pax 3 (Cat. No. Mm00435493_m1); all values were normalized using the Mouse GAPD (GAPDH) Endogenous Control (VIC®/MGB Probe, Primer Limited, Invitrogen, Cat. No. 4352339E). As it can be seen in FIG. 3, all three early myogenic markers, which are neither expressed in primary fibroblasts nor in undifferentiated iPSM cells (iPSM not cultured in the myogenic differentiation medium), can be detected in iPSM cells differentiated with above protocol. Altogether, these results suggest that the cells correspond to myogenic progenitors that did not yet differentiate into myoblasts.

To address the differentiation of iPSM cells into myofibers, we co-cultured iPSM cells with C2C12 cells. Cells were split on day 14 onto sub-confluent C2C12 cells (without matrigel) and cultured as follows:
1 day [day 15] in growth medium supplemented with 1 uM retinoic acid and 10 uM 5-Azacytidine.
1 day [day 16] in growth medium supplemented with 1 uM RA, 100 ng/ml HGF, 2 ng/ml bFGF, 2 ng/ml IGF1, 10 ng/ml Wnt4, and 1 ug/ml Shh or 200 nM SAG.
2 days [days 17-18] in growth medium supplemented with 1 uM RA, 100 ng/ml HGF, 2 ng/ml bFGF, 2 ng/ml IGF1, and 1 ug/ml Shh or 200 nM SAG.
2 days in DMEM/F12 (1:1), Invitrogen, supplemented with 2% horse serum
6 days in DMEM LOW GLUCOSE (Invitrogen), supplemented with 20% FBS.

To identify iPSM-derived cells, we used iPSM cells carrying a transgene expressing LacZ under a ubiquitous promoter, thereby allowing a fluorescent labeling of iPSM derived cells/myofibers (ImageGene Green C12FDB lacZ Gene Expression Kit, Invitrogen, Cat. No. 1-2904).
Myofibers (labeled by MF20 immunostaining), to which iPSM cells contributed, could be observed (data not shown), suggesting that iPSM cells are capable of differentiating into myoblasts and fusing with C2C12 cells in order to give rise to myofibers.

### Example 6

### Chondrogenic Differentiation

iPSM cells were FACS sorted and plated on fibronectin-coated 48-well tissue culture plates. Cells were grown in growth medium as described above. After 7 days, the medium was supplemented with 10 ng/ul Wnt3a in the presence of doxycycline. On day 14, when approximately 25% of the cells expressed the reporter at high levels, cells were split onto fibronectin-coated 48-well tissue culture plates and cultured as follows:
1 day [day 15] in growth medium supplemented with 1 uM retinoic acid and 10 uM 5-Azacytidine.
3 days [days 16-18] in growth medium supplemented with 100 ng/ml Bmp4 (R&D systems, Cat. No. 5020-BP-010).

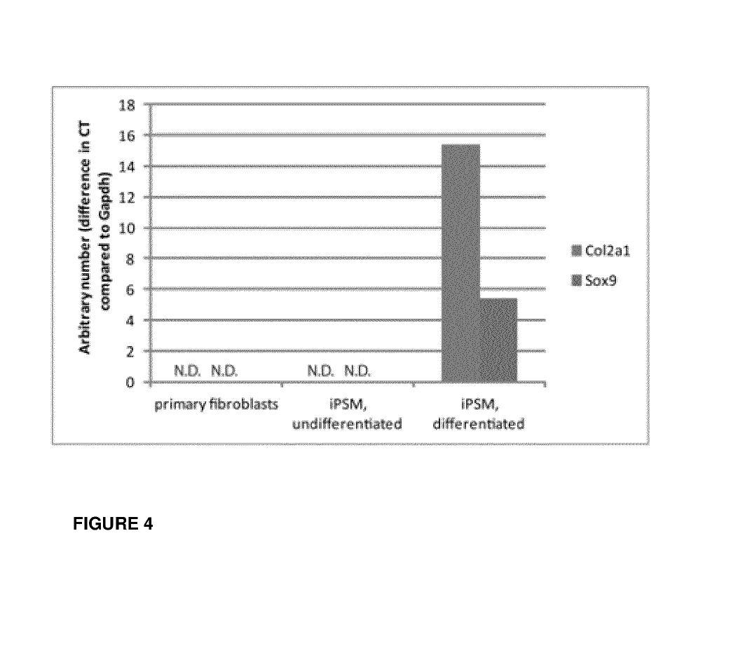

RNA was extracted using Trizol and one-step quantitative real-time PCR was performed to measure the expression of Sox9 and Col2A1 as described previously (Taqman probes Applied Biosystems, Cat. No. Mm00448840_m1 for Sox9 and Cat. No. Mm01309565_m1for Col2A1). As it can be seen in FIG. 4, a robust amplification of Sox9 and Col2a1 is observed in iPSM differentiated according to the chondrogenic protocol described above, whereas no expression was detected in primary fibroblasts or in undifferentiated iPSM cells.

To confirm this observation, we further cultured iPSM cells with above protocol, followed by 2 days with the STEMPRO Chondrogenesis Differentiation Kit (Invitrogen, Cat. No. A10071-01), and SOX9 protein could be detected by immunohistochemistry (Anti SOX9 Santa Cruz Biotechnology (H-90) Rabbit IgG, Cat. No. SC-20095) (Data not shown).

### Example 7

### Reprogramming of Human Fibroblasts

In order to identify human iPSM cells with a fluorescent reporter, we generated a lentivirus-based reporter by cloning the human MSGN1 promoter (6.8 kb genomic sequence upstream of start codon) in front of the coding sequence of Venus (in between cPPT and WRPE of pLVX vector) (pLVX-HsMSGN1-Venus). We additionally constructed a pLVX-tight-puro based Lentivirus where a bicistronic human T—IRES2-dnRAR construct (pLVX-tight-HsT-IRES2-dnRAR-puro) was cloned into the multiple cloning site; the sequence used for human T contains the two exons present in the EnsEMBL/Havanna sequence but absent in the NCBI RefSeq sequence.

As target cells, we selected primary human dermal neonatal fibroblasts (Invitrogen, Cat. No. C0045C). After 2 passages, cells were co-infected at 80% confluence with HsT-IRES2-dnRAR and tet-off-advanced containing lentiviral particles, followed by a second infection on the following day with HsMSGN-Venus containing lentiviral particles. Venus positive cells were observed after 4 weeks of culture (Data not shown).

### Example 8

### Useful Nucleotide and Amino Acid Sequences for Practicing the Methods of the Invention

TABLE 3

| NO: | Description | Sequence |
|---|---|---|
| 1 | Human Brachyury | MSSPGTESAGKSLQYRVDHLLSAVENELQAGSEK |
| | amino acid sequence | GDPTERELRVGLEESELWLRFKELTNEMIVTKNGR |
| | (NP_003172) | RMFPVLKVNVSGLDPNAMYSFLLDFVAADNHRW |
| | | KYVNGEWVPGGKPEPQAPSCVYIHPDSPNFGAHW |
| | | MKAPVSFSKVKLTNKLNGGGQIMLNSLHKYEPRIH |
| | | IVRVGGPQRMITSHCFPETQFIAVTAYQNEEITALKI |
| | | KYNPFAKAFLDAKERSDHKEMMEEPGDSQQPGYS |
| | | QWGWLLPGTSTLCPPANPHPQFGGALSLPSTHSCD |
| | | RYPTLRSHRSSPYPSPYAHRNNSPTYSDNSPACLSM |
| | | LQSHDNWSSLGMPAHPSMLPVSHNASPPTSSSQYP |
| | | SLWSVSNGAVTPGSQAAAVSNGLGAQFFRGSPAH |
| | | YTPLTHPVSAPSSSGSPLYEGAAAATDIVDSQYDA |
| | | AAQGRLIASWTPVSPPSM |
| 2 | Mouse Brachyury | MSSPGTESAGKSLQYRVDHLLSAVESELQAGSEKG |
| | amino acid sequence | DPTERELRVGLEESELWLRFKELTNEMIVTKNGRR |
| | (NP_033335) | MFPVLKVNVSGLDPNAMYSFLLDFVTADNHRWK |
| | | YVNGEWVPGGKPEPQAPSCVYIHPDSPNFGAHWM |
| | | KAPVSFSKVKLTNKLNGGGQIMLNSLHKYEPRIHI |
| | | VRVGGPQRMITSHCFPETQFIAVTAYQNEEITALKI |
| | | KYNPFAKAFLDAKERNDHKDVMEEPGDCQQPGY |
| | | SQWGWLVPGAGTLCPPASSHPQFGGSLSLPSTHGC |
| | | ERYPALRNHRSSPYPSPYAHRNSSPTYADNSSACLS |
| | | MLQSHDNWSSLGVPGHTSMLPVSHNASPPTGSSQ |
| | | YPSLWSVSNGTITPGSQTAGVSNGLGAQFFRGSPA |
| | | HYTPLTHTVSAATSSSSGSPMYEGAATVTDISDSQ |
| | | YDTAQSLLIASWTPVSPPSM |
| 3 | Human Brachyury | ATGAGCTCCCCTGGCACCGAGAGCGCGGGAAAG |
| | coding sequence | AGCCTGCAGTACCGAGTGGACCACCTGCTGAGC |
| | (NM_003181) | GCCGTGGAGAATGAGCTGCAGGCGGGCAGCGAG |
| | | AAGGGCGACCCCACAGAGCGCGAACTGCGCGTG |
| | | GGCCTGGAGGAGAGCGAGCTGTGGCTGCGCTTC |
| | | AAGGAGCTCACCAATGAGATGATCGTGACCAAG |
| | | AACGGCAGGAGGATGTTTCCGGTGCTGAAGGTG |
| | | AACGTGTCTGGCCTGGACCCCAACGCCATGTACT |
| | | CCTTCCTGCTGGACTTCGTGGCGGCGGACAACCA |
| | | CCGCTGGAAGTACGTGAACGGGGAATGGGTGCC |
| | | GGGGGGCAAGCCGGAGCCGCAGGCGCCCAGCTG |
| | | CGTCTACATCCACCCCGACTCGCCCAACTTCGGG |
| | | GCCCACTGGATGAAGGCTCCCGTCTCCTTCAGCA |
| | | AAGTCAAGCTCACCAACAAGCTCAACGGAGGGG |
| | | GCCAGATCATGCTGAACTCCTTGCATAAGTATGA |
| | | GCCTCGAATCCACATAGTGAGAGTTGGGGGTCC |
| | | ACAGCGCATGATCACCAGCCACTGCTTCCCTGA |
| | | GACCCAGTTCATAGCGGTGACTGCTTATCAGAA |
| | | CGAGGAGATCACAGCTCTTAAAATTAAGTACAA |
| | | TCCATTTGCAAAAGCTTTCCTTGATGCAAAGGAA |
| | | AGAAGTGATCACAAAGAGATGATGGAGGAACCC |
| | | GGAGACAGCCAGCAACCTGGGTACTCCCAATGG |
| | | GGGTGGCTTCTTCCTGGAACCAGCACCCTGTGTC |
| | | CACCTGCAAATCCTCATCCTCAGTTTGGAGGTGC |
| | | CCTCTCCCTCCCCTCCACGCACAGCTGTGACAGG |
| | | TACCCAACCCTGAGGAGCCACCGGTCCTCACCCT |
| | | ACCCCAGCCCCTATGCTCATCGGAACAATTCTCC |
| | | AACCTATTCTGACAACTCACCTGCATGTTTATCC |
| | | ATGCTGCAATCCCATGACAATTGGTCCAGCCTTG |
| | | GAATGCCTGCCCATCCCAGCATGCTCCCCGTGAG |
| | | CCACAATGCCAGCCCACCTACCAGCTCCAGTCA |
| | | GTACCCCAGCCTGTGGTCTGTGAGCAACGGCGC |
| | | CGTCACCCCGGGCTCCCAGGCAGCAGCCGTGTC |
| | | CAACGGGCTGGGGGCCCAGTTCTTCCGGGGCTC |
| | | CCCCGCGCACTACACACCCCTCACCCATCCGGTC |
| | | TCGGCGCCCTCTTCCTCGGGATCCCCACTGTACG |
| | | AAGGGGCGGCCGCGGCCACAGACATCGTGGACA |
| | | GCCAGTACGACGCCGCAGCCCAAGGCCGCCTCA |
| | | TAGCCTCATGGACACCTGTGTCGCCACCTTCCAT |
| | | GTGA |
| 4 | Mouse Brachyruy | ATGAGCTCGCCGGGCACAGAGAGCGCAGGGAA |
| | coding sequence | GAGCCTGCAGTACCGAGTGGACCACCTGCTCAG |
| | (NM_009309) | CGCCGTGGAGAGCGAGCTGCAGGCGGGCAGCGA |
| | | GAAGGGAGACCCCACCGAACGCGAACTGCGAGT |
| | | GGGCCTGGAGGAGAGCGAGCTGTGGCTGCGCTT |
| | | CAAGGAGCTAACTAACGAGATGATTGTGACCAA |
| | | GAACGGCAGGAGGATGTTCCCGGTGCTGAAGGT |
| | | AAATGTGTCAGGCCTGGACCCCAATGCCATGTA |
| | | CTCTTTCTTGCTGGACTTCGTGACGGCTGACAAC |
| | | CACCGCTGGAAATATGTGAACGGGGAGTGGGTA |
| | | CCTGGGGGCAAACCAGAGCCTCAGGCGCCCAGC |
| | | TGCGTCTACATCCACCCAGACTCGCCCAATTTTG |
| | | GGGCCCACTGGATGAAGGCGCCTGTGTCTTTCA |
| | | GCAAAGTCAAACTCACCAACAAGCTCAATGGAG |
| | | GGGGACAGATCATGTTAAACTCCTTGCATAAGT |
| | | ATGAACCTCGGATTCACATCGTGAGAGTTGGGG |
| | | GCCCGCAACGCATGATCACCAGCCACTGCTTTCC |
| | | CGAGACCCAGTTCATAGCTGTGACTGCCTACCA |
| | | GAATGAGGAGATTACAGCCCTTAAAATTAAATA |
| | | CAACCCATTTGCTAAAGCCTTCCTTGATGCCAAA |
| | | GAAAGAAACGACCACAAAGATGTAATGGAGGA |
| | | ACCGGGGGACTGCCAGCAGCCGGGGTATTCCCA |
| | | ATGGGGGTGGCTTGTTCCTGGTGCTGGCACCCTC |
| | | TGCCCGCCTGCCAGCTCCCACCCTCAGTTTGGAG |
| | | GCTCGCTCTCTCTCCCCTCCACACACGGCTGTGA |
| | | GAGGTACCCAGCTCTAAGGAACCACCGGTCATC |
| | | GCCCTACCCCAGCCCCTATGCTCATCGGAACAGC |
| | | TCTCCAACCTATGCGGACAATTCATCTGCTTGTC |
| | | TGTCCATGCTGCAGTCCCATGATAACTGGTCTAG |
| | | CCTCGGAGTGCCTGGCCACACCAGCATGCTGCCT |
| | | GTGAGTCATAACGCCAGCCCACCTACTGGCTCTA |
| | | GCCAGTATCCCAGTCTCTGGTCTGTGAGCAATGG |
| | | TACCATCACCCCAGGCTCCCAGACAGCTGGGGT |
| | | GTCCAACGGGCTGGGAGCTCAGTTCTTTCGAGG |
| | | CTCCCCTGCACATTACACACCACTGACGCACACG |
| | | GTCTCAGCTGCCACGTCCTCGTCTTCTGGTTCTC |
| | | CGATGTATGAAGGGGCTGCTACAGTCACAGACA |
| | | TTTCTGACAGCCAGTATGACACGGCCCAAAGCC |
| | | TCCTCATAGCCTCGTGGACACCTGTGTCACCCCC |
| | | ATCTATGTGA |
| 5 | Human dnRAR coding | ATGGCCAGCAACAGCAGCTCCTGCCCGACACCT |
| | sequence | GGGGGCGGGCACCTCAATGGGTACCCGGTGCCT |
| | (experimentally | CCCTACGCCTTCTTCTTCCCCCCTATGCTGGGTG |
| | obtained construct, no | GACTCTCCCCGCCAGGCGCTCTGACCACTCTCCA |
| | GenBank accession | GCACCAGCTTCCAGTTAGTGGATATAGCACACC |
| | number) | ATCCCCAGCCACCATTGAGACCCAGAGCAGCAG |
| | | TTCTGAAGAGATAGTGCCCAGCCCTCCCTCGCCA |
| | | CCCCCTCTACCCCGCATCTACAAGCCTTGCTTTG |
| | | TCTGTCAGGACAAGTCCTCAGGCTACCACTATGG |
| | | GGTCAGCGCCTGTGAGGGCTGCAAGGGCTTCTT |
| | | CCGCCGCAGCATCCAGAAGAACATGGTGTACAC |
| | | GTGTCACCGGGACAAGAACTGCATCATCAACAA |
| | | GGTGACCCGGAACCGCTGCCAGTACTGCCGACT |
| | | GCAGAAGTGCTTTGAAGTGGGCATGTCCAAGGA |
| | | GTCTGTGAGAAACGACCGAAACAAGAAGAAGA |
| | | AGGAGGTGCCCAAGCCCGAGTGCTCTGAGAGCT |
| | | ACACGCTGACGCCGGAGGTGGGGGAGCTCATTG |
| | | AGAAGGTGCGCAAAGCGCACCAGGAAACCTTCC |
| | | CTGCCCTCTGCCAGCTGGGCAAATACACTACGA |
| | | ACAACAGCTCAGAACAACGTGTCTCTCTGGACA |
| | | TTGACCTCTGGGACAAGTTCAGTGAACTCTCCAC |
| | | CAAGTGCATCATTAAGACTGTGGAGTTCGCCAA |
| | | GCAGCTGCCCGGCTTCACCACCCTCACCATCGCC |
| | | GACCAGATCACCCTCCTCAAGGCTGCCTGCCTGG |
| | | ACATCCTGATCCTGCGGATCTGCACGCGGTACAC |
| | | GCCCGAGCAGGACACCATGACCTTCTCGGACGG |
| | | GCTGACCCTGAACCGGACCCAGATGCACAACGC |
| | | TGGCTTCGGCCCCCTCACCGACCTGGTCTTTGCC |
| | | TTCGCCAACCAGCTGCTGCCCCTGGAGATGGAT |
| | | GATGCGGAGACGGGGCTGCTCAGCGCCATCTGC |
| | | CTCATCTGCGGAGACCGCCAGGACCTGGAGCAG |
| | | CCGGACCGGGTGGACATGCTGCAGGAGCCGCTG |
| | | CTGGAGGCGCTAAAGGTCTACGTGCGGAAGCGG |
| | | AGGCCCAGCCGCCCCCACATGTTCCCCAAGATG |
| | | CTAATGAAGATTACTGACCTGCGAAGCATCAGC |
| | | GCCAAGGGGGCTGAGCGGGTGATCACGCTGAAG |
| | | ATGGAGATCCCATCAGGATCCTGGCCAGCTAGC |
| | | TAG |
| 6 | Mouse dnRAR | ATGGCCAGCAATAGCAGTTCCTGCCCAACACCT |
| | codingsequence | GGGGGCGGGCACCTCAATGGGTACCCAGTACCC |
| | | CCCTACGCCTTCTTCTTTCCCCCTATGCTGGGTG |
| | | GACTCTCCCCACCCGGCGCTCTCACCAGCCTCCA |
| | | GCACCAGCTTCCAGTCAGTGGTTACAGCACACC |
| | | GTCCCCAGCCACCATCGAGACCCAGAGCAGCAG |
| | | TTCCGAAGAGATAGTACCCAGCCCTCCCTCACCA |
| | | CCGCCCCTGCCCCGCATCTACAAGCCTTGCTTTG |
| | | TTTGTCAAGACAAATCATCCGGCTACCACTATGG |
| | | GGTCAGCGCCTGTGAGGGCTGTAAGGGCTTCTTC |
| | | CGACGAAGCATCCAGAAGAACATGGTGTATACG |
| | | TGTCACCGGGACAAGAACTGCATCATCAACAAG |
| | | GTGACCCGGAACCGCTGCCAGTACTGCCGGCTG |
| | | CAGAAATGTTTCGACGTGGGCATGTCCAAGGAG |
| | | TCGGTGCGAAACGATCGAAACAAAAAGAAGAA |
| | | AGAGGCACCCAAGCCCGAGTGCTCAGAGAGCTA |
| | | CACGCTGACGCCTGAGGTGGGCGAGCTCATTGA |
| | | GAAGGTTCGCAAAGCGCACCAGGAGACCTTCCC |
| | | GGCCCTCTGCCAGCTGGGCAAGTACACTACGAA |
| | | CAACAGCTCAGAACAACGAGTCTCCCTGGACAT |
| | | TGACCTCTGGGACAAGTTCAGTGAACTCTCCACC |
| | | AAGTGCATCATTAAGACTGTGGAGTTCGCCAAG |
| | | CAGCTTCCCGGCTTCACCACCCTCACCATCGCCG |
| | | ACCAGATCACCCTCCTCAAGGCTGCCTGCCTGGA |
| | | TATCCTGATTCTGCGAATCTGCACGCGGTACACG |
| | | CCTGAGCAAGACACAATGACCTTCTCAGATGGA |
| | | CTGACCCTGAACCGGACTCAGATGCACAACGCT |
| | | GGCTTTGGCCCCCTCACCGACTTGGTCTTTGCCT |
| | | TCGCCAACCAGCTGCTGCCCCTGGAGATGGACG |
| | | ATGCTGAGACTGGACTGCTCAGTGCCATCTGCCT |
| | | CATCTGTGGAGACCGACAGGACCTGGAGCAGCC |
| | | AGACAAGGTGGACATGCTGCAAGAGCCGCTGCT |
| | | GGAAGCACTGAAAGTCTACGTCCGGAAACGGAG |
| | | GCCCAGCCGACCCCACATGTTCCCCAAGATGCT |
| | | GATGAAGATCACAGACCTTCGGAGCATCAGCGC |
| | | CAAGGGAGCTGAACGGGTGATCACATTGAAGAT |
| | | GGAGATCCCATAA |
| 7 | Human Msgn1 coding | ATGGACAACCTGCGCGAGACTTTCCTCAGCCTCG |
| | sequence | AGGATGGCTTGGGCTCCTCTGACAGCCCTGGCCT |
| | (NM_001105569) | GCTGTCTTCCTGGGACTGGAAGGACAGGGCAGG |
| | | GCCCTTTGAGCTGAATCAGGCCTCCCCCTCTCAG |
| | | AGCCTTTCCCCGGCTCCATCGCTGGAATCCTATT |
| | | CTTCTTCTCCCTGTCCAGCTGTGGCTGGGCTGCC |
| | | CTGTGAGCACGGCGGGGCCAGCAGTGGGGGCAG |
| | | CGAAGGCTGCAGTGTCGGTGGGGCCAGTGGCCT |
| | | GGTAGAGGTGGACTACAATATGTTAGCTTTCCA |
| | | GCCCACCCACCTTCAGGGCGGTGGTGGCCCCAA |
| | | GGCCCAGAAGGGCACCAAAGTCAGGATGTCTGT |
| | | CCAGCGGAGGCGGAAAGCCAGCGAGAGGGAGA |
| | | AGCTCAGGATGAGGACCTTGGCAGATGCCCTGC |
| | | ACACCCTCCGGAATTACCTGCCACCTGTCTACAG |
| | | CCAGAGAGGCCAGCCTCTCACCAAGATCCAGAC |
| | | ACTCAAGTACACCATCAAGTACATCGGGGAACT |
| | | CACAGACCTCCTTAACCGCGGCAGAGAGCCCAG |
| | | AGCCCAGAGCGCGTGA |
| 8 | Mouse Msgn1 coding | ATGGACAACCTGGGTGAGACCTTCCTCAGCCTG |
| | sequence | GAGGATGGCCTGGACTCTTCTGACACCGCTGGTC |
| | (NM_019544) | TGCTGGCCTCCTGGGACTGGAAAAGCAGAGCCA |
| | | GGCCCTTGGAGCTGGTCCAGGAGTCCCCCACTC |
| | | AAAGCCTCTCCCCAGCTCCTTCTCTGGAGTCCTA |
| | | CTCTGAGGTCGCACTGCCCTGCGGGCACAGTGG |
| | | GGCCAGCACAGGAGGCAGCGATGGCTACGGCAG |
| | | TCACGAGGCTGCCGGCTTAGTCGAGCTGGATTA |
| | | CAGCATGTTGGCTTTTCAACCTCCCTATCTACAC |
| | | ACTGCTGGTGGCCTCAAAGGCCAGAAAGGCAGC |
| | | AAAGTCAAGATGTCTGTCCAGCGGAGACGGAAG |
| | | GCCAGCGAGAGAGAGAAACTCAGGATGCGGAC |
| | | CTTAGCCGATGCCCTCCACACGCTCCGGAATTAC |
| | | CTGCCGCCTGTCTACAGCCAGAGAGGCCAACCG |
| | | CTCACCAAGATCCAGACACTCAAGTACACCATC |
| | | AAGTACATCGGGGAACTCACAGACCTCCTCAAC |
| | | AGCAGCGGGAGAGAGCCCAGGCCACAGAGTGT |
| | | GTGA |
| 9 | CDS of human T used | atgagctcccctggcaccgagagcgcgggaaagagcctgcagtaccgagtgg |
| | for HsT-IRES2-dnRAR | accacctgctgagcgccgtggagaatgagctgcaggcgggcagcgagaagg |
| | construct | gcgaccccacagagcgcgaactgcgcgtgggcctggaggagagcgagctgt |
| | | ggctgcgcttcaaggagctcaccaatgagatgatcgtgaccaagaacggcagg |
| | | aggatgtttccggtgctgaaggtgaacgtgtctggcctggaccccaacgccatg |
| | | tactccttcctgctggacttcgtggcggcggacaaccaccgctggaagtacgtg |
| | | aacggggaatgggtgccggggggcaagccggagccgcaggcgcccagctg |
| | | cgtctacatccaccccgactcgcccaacttcggggcccactggatgaaggctcc |
| | | cgtctccttcagcaaagtcaagctcaccaacaagctcaacggagggggccaga |
| | | tcatgctgaactccttgcataagtatgagcctcgaatccacatagtgagagttggg |
| | | ggtccacagcgcatgatcaccagccactgcttccctgagacccagttcatagcg |
| | | gtgactgcttatcagaacgaggagatcacagctcttaaaattaagtacaatccatt |
| | | tgcaaaagctttccttgatgcaaaggaaagaagtgatcacaaagagatgatgga |
| | | ggaacccggagacagccagcaacctgggtactcccaatgggggtggcttcttc |
| | | ctggaaccagcaccctgtgtccacctgcaaatcctcatcctcagtttggaggtgc |
| | | cctctccctcccctccacgcacagctgtgacaggtacccaaccctgaggagcc |
| | | accggtcctcaccctaccccagcccctatgctcatcggaacaattctccaaccta |
| | | ttctgacaactcacctgcatgtttatccatgctgcaatcccatgacaattggtccag |
| | | ccttggaatgcctgcccatcccagcatgctccccgtgagccacaatgccagccc |
| | | acctaccagctccagtcagtaccccagcctgtggtctgtgagcaacggcgccgt |
| | | caccccgggctcccaggcagcagccgtgtccaacgggctgggggcccagttc |
| | | ttccggggctcccccgcgcactacacacccctcacccatccggtctcggcgcc |
| | | ctcttcctcgggatccccactgtacgaaggggcggccgcggccacagacatcg |
| | | tggacagccagtacgacgccgcagcccaaggccgcctcatagcctcatggac |
| | | acctgtgtcgccaccttccatgtga |

### REFERENCES

Agulnik, S. I., Garvey, N., Hancock, S., Ruvinsky, I., Chapman, D. L., Agulnik, I., Bollag, R., Papaioannou, V., and Silver, L. M. 1996. Evolution of mouse T-box genes by tandem duplication and cluster dispersion. Genetics 144(1): 249-254.
Chambers, I., and Smith, A. 2004. Self-renewal of teratocarcinoma and embryonic stem cells. Oncogene 23: 7150-7160.
Chapman, D. L., Agulnik, I., Hancock, S., Silver, L. M., and Papaioannou, V. E. 1996a. Tbx6, a mouse T-Box gene implicated in paraxial mesoderm formation at gastrulation. Dev Biol 180(2): 534-542.
Chapman, D. L., Garvey, N., Hancock, S., Alexiou, M., Agulnik, S. I., Gibson-Brown, J. J., Cebra-Thomas, J., Bollag, R. J., Silver, L. M., and Papaioannou, V. E. 1996b. Expression of the T-box family genes, Tbx1-Tbx5, during early mouse development. Dev Dyn 206(4): 379-390.
Chapman, D. L. and Papaioannou, V. E. 1998. Three neural tubes in mouse embryos with mutations in the T-box gene Tbx6. Nature 391: 695-697.
Conlon F. L., Sedgwick S. G., Weston K. M., and Smith J. C. 1996. Inhibition of Xbra transcription activation causes defects in mesodermal patterning and reveals autoregulation of Xbra in dorsal mesoderm. Development 122(8): 2427-35
Damm K., Heyman R. A., Umesono K., Evans R. M. 1993. Functional inhibition of retinoic acid response by dominant negative retinoic acid receptor mutants. Proc. Natl. Acad. Sci. USA 90: 2989-2993.
Dimos, J. T., Rodolfa, K. T., Niakan, K. K., Weisenthal, L. M., Mitsumoto, H., Chung, W., Croft, G. F., Saphier, G., Leibel, R., Goland, R. et al. 2008. Induced pluripotent stem cells generated from patients with ALS can be differentiated into motor neurons. Science 321(5893): 1218-1221.
Dinser R., Pelled G., Müller-Ladner U., Gazit D., and Neumann E. 2009. Expression of Brachyury in mesenchymal progenitor cells leads to cartilage-like tissue that is resistant to the destructive effect of rheumatoid arthritis synovial fibroblasts. J Tissue Eng Regen Med. 3(2): 124-8.
Dinsmore, J., Ratliff, J., Deacon, T., Pakzaban, P., Jacoby, D., Galpern, W., and Isacson, O. 1996. Embryonic stem cells differentiated in vitro as a novel source of cells for transplantation. Cell transplantation 5(2): 131-143.
Gadue, P., Huber, T. L., Paddison, P. J., and Keller, G. M. 2006. Wnt and TGF-beta signaling are required for the induction of an in vitro model of primitive streak formation using embryonic stem cells. Proc Natl Acad Sci USA 103(45): 16806-16811.
Harada N., Tamai Y., Ishikawa T., Sauer B., Takaku K., Oshima M., and Taketo M. M. 1999. Intestinal polyposis in mice with a dominant stable mutation of the bold beta-catenin gene Embo J 18(21):5931-5942
Hoffmann A., Czichos S., Kaps C., Bächner D., Mayer H., Kurkalli B. G., Zilberman Y., Turgeman G., Pelled G., Gross G., and Gazit D. 2002. The T-box transcription factor
Brachyury mediates cartilage development in mesenchymal stem cell line C3H10T1/2. J Cell Sci. 115(4):769-81
Hug, B., Walter, V., and Grunwald, D. J. 1997. tbx6, a Brachyury-related gene expressed by ventral mesendodermal precursors in the zebrafish embryo. Dev Biol 183(1): 61-73.
Ieda, M., Fu, J. D., Delgado-Olguin, P., Vedantham, V., Hayashi, Y., Bruneau, B. G., and Srivastava, D. 2010. Direct reprogramming of fibroblasts into functional cardiomyocytes by defined factors. Cell 142(3): 375-386.
Kispert, A. and Hermann, B. G. 1993. The Brachyury gene encodes a novel DNA binding protein. Embo J 12(12): 4898-4899.
Knezevic, V., De Santo, R., and Mackem, S. 1997. Two novel chick T-box genes related to mouse Brachyury are expressed in different, non-overlapping mesodermal domains during gastrulation. Development 124(2): 411-419.
Mansour, S. J., Matten, W. T., Hermann, A. S., Candia, J. M., Rong, S., Fukasawa, K., Vande Woude, G. F., and Ahn, N. G. 1994. Transformation of mammalian cells by constitutively active MAP kinase kinase. Science 265(5174):966-970
Metallo C., Mohr J., Detzel C., de Pablo J. J., Van Wie B. J., and Palecek S. P. 2007. Engineering the stem cell microenvironment. Biotechnol Prog 23:18-23.
Myers, E. W., and Miller, W. 1988. Optimal alignments in linear space. Comput. Appl. Biosci. 4:11-17.
Park, I. H., Arora, N., Huo, H., Maherali, N., Ahfeldt, T., Shimamura, A., Lensch, M. W., Cowan, C., Hochedlinger, K., and Daley, G. Q. 2008a. Disease-specific induced pluripotent stem cells. Cell 134(5): 877-886.
Park, I. H., Zhao, R., West, J. A., Yabuuchi, A., Huo, H., Ince, T. A., Lerou, P. H., Lensch, M. W., and Daley, G. Q. 2008b. Reprogramming of human somatic cells to pluripotency with defined factors. Nature 451(7175): 141-146.
Rashbass, P., Wilson, V., Rosen, B., and Beddington, R. S. 1994. Alterations in gene expression during mesoderm formation and axial patterning in Brachyury (T) embryos. Int J Dev Biol 38(1): 35-44.
Takahashi, K., Tanabe, K., Ohnuki, M., Narita, M., Ichisaka, T., Tomoda, K., and Yamanaka, S. 2007. Induction of pluripotent stem cells from adult human fibroblasts by defined factors. Cell 131(5): 861-872.
Takahashi, K. and Yamanaka, S. 2006. Induction of pluripotent stem cells from mouse embryonic and adult fibroblast cultures by defined factors. Cell 126(4): 663-676.
Vierbuchen, T., Ostermeier, A., Pang, Z. P., Kokubu, Y., Sudhof, T. C., and Wernig, M. 2010. Direct conversion of fibroblasts to functional neurons by defined factors. Nature 463(7284): 1035-1041.
Warren L, Manos P. D., Ahfeldt T., Loh Y. H., Li H., Lau F., Ebina W., Mandal P. K., Smith Z. D., Meissner A., Daley G. Q., Brack A. S., Collins J. J., Cowan C., Schlaeger T. M., and Rossi D. J. 2010. Highly efficient reprogramming to pluripotency and directed differentiation of human cells with synthetic modified mRNA. Cell Stem Cell. November 5; 7(5):618-30.
Weintraub, H., Tapscott, S. J., Davis, R. L., Thayer, M. J., Adam, M. A., Lassar, A. B., and Miller, A. D. 1989. Activation of muscle-specific genes in pigment, nerve, fat, liver, and fibroblast cell lines by forced expression of MyoD. Proc Natl Acad Sci USA 86(14): 5434-5438.
Wilkinson, D. G., Bhatt, S., and Herrmann, B. G. 1990. Expression pattern of the mouse T gene and its role in mesoderm formation. Nature 343(6259): 657-659.
Wittler, L., Shin, E. H., Grote, P., Kispert, A., Beckers, A., Gossler, A., Werber, M., and Herrmann, B. G. 2007. Expression of Msgn1 in the presomitic mesoderm is controlled by synergism of WNT signalling and Tbx6. EMBO Rep 8(8): 784-789.
Yamaguchi, T. P., Takada, S., Yoshikawa, Y., Wu, N., and McMahon, A. P. 1999. T (Brachyury) is a direct target of Wnt3a during paraxial mesoderm specification. Genes Dev 13(24): 3185-3190.
Yoon, J. K., Moon, R. T., and Wold, B. 2000. The bHLH class protein pMesogenin1 can specify paraxial mesoderm phenotypes [In Process Citation]. Dev Biol 222(2): 376-391.
Yoon, J. K. and Wold, B. 2000. The bHLH regulator pMesogenin1 is required for maturation and segmentation of paraxial mesoderm. Genes Dev 14(24): 3204-3214.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R. et al. 2007. Induced pluripotent stem cell lines derived from human somatic cells. Science 318(5858): 1917-1920.
Zhou, Q., Brown, J., Kanarek, A., Rajagopal, J., and Melton, D. A. 2008. In vivo reprogramming of adult pancreatic exocrine cells to beta-cells. Nature 455(7213): 627-632.
Throughout this application, various references describe the state of the art to which this invention pertains. The disclosures of these references are hereby incorporated by reference into the present disclosure.

1. An ex vivo method for preparing induced presomitic mesoderm (iPSM) cells, said method comprising the steps of:
a) providing target cells to be reprogrammed, and,
b) culturing said target cells under appropriate conditions for reprogramming said cells into iPSM cells, wherein said appropriate conditions comprise increasing expression of at least Brachyury transcription factor in said cells.

2. The method of claim 1, wherein said target cells are primary cells.

3. The method of claim 1, wherein said induced presomitic mesoderm (iPSM) cells have long-term self renewal properties and are capable of differentiating into at least skeletal, dermis or muscle cell lineages.

4. The method of claim 1, further comprising the following step c) of detecting or selecting among the cultured cells, those expressing one or more of the biomarkers characteristic of presomitic mesoderm cells.

5. The method of claim 1, wherein said appropriate conditions for reprogramming said target cells into iPSM cells further comprise inhibiting at least retinoic acid signalling in said target cells.

6. The method according to claim 5, wherein conditions for inhibiting retinoic acid signalling are selected from the group consisting of:
a) allowing ectopic expression of a nucleic acid construct encoding a dominant negative retinoic acid receptor (dnRAR) in said target cells,
b) culturing the target cells with an appropriate amount of one or more compound inhibitors of retinoic acid receptor signalling or of retinaldehyde dehydrogenase,
c) culturing the target cells in medium depleted in retinoids,
d) inhibiting expression of a gene involved in retinoic acid signalling in said target cells and
e) overexpressing a protein involved in retinoic acid catabolism in said target cells.

7. The method according to claim 1, wherein said conditions for increasing expression of at least Brachyrury transcription factor comprise either
a) introducing an expression vector comprising the gene encoding said Brachyury transcription factor into said target cells;
b) modulating Wnt, BMP or FGF signalling; and/or,
c) introducing an effective amount of Brachyruy transcription factor or its precursor RNA into said target cells.

8. The method according to claim 1, wherein
a) said conditions for increasing expression of Brachyury transcription factor comprise the direct introduction of the Brachyury transcription factor into said target cells in an amount sufficient for auto-induction of the expression of endogenous Brachyury transcription factor; and,
b) said appropriate conditions for reprogramming the cells into iPSM cells further comprise culturing the target cells in the presence of one or more inhibitors of retinoic acid signalling; and,
said method does not involve any genetic modification of said target cells.

9. A composition comprising iPSM cells obtainable from the method of claim 1, characterized in that at least 10%, 20%, 30%, 40%, 50%, 60%, 70%, 80% or 90% of the cells in said composition, exhibit a high expression of biomarker characteristic of presomitic mesoderm cells.

10. A method for preparing compositions comprising skeletal muscle, bone, cartilage or dermal cell lineages, said method comprising the steps of
a) providing a composition comprising iPSM cells according to claim 9; and
b) culturing said composition comprising iPSM cells, under appropriate conditions for their differentiation into a desired cell lineage selected from the group consisting of the group consisting of skeletal muscle, bone, cartilage, or dermal cells.

11. The method according to claim 10, for preparing compositions comprising skeletal muscle cell lineages, said method comprising the steps of
a) providing a composition comprising iPSM cells;
b) culturing said composition comprising iPSM cells in the presence of a differentiation medium comprising at least the following components:
i. an extracellular matrix material; and,
ii. compounds activating or inhibiting the signalling pathways known to control the differentiation of said lineages, said signalling pathways being selected from the group consisting of retinoic acid, BMP, Hedgehog, Notch, FGF, Wnt, myostatin, insulin, PDGF, MAPK and PI3K, DNA methylation, DNA acetylation; and,
c) optionally, culturing said composition obtained from step (b) in a second differentiation medium comprising at least one or more of the differentiation factors selected from the group consisting of bFGF, HGF, horse serum, Activin A, transferrin, EGF, Insulin, LiCl, and IGF-1,
thereby obtaining a composition comprising skeletal muscle cell lineages.

12. The method according to claim 10, for preparing a composition comprising dermal cell lineages, said method comprising the step of culturing a composition comprising iPSM cells in the presence of an efficient amount of at least one or more of the differentiation factors selected from the group consisting of BMP, Wnt, FGF, EGF, retinoic acid, and Hedgehog families of growth factors.

13. The method according to claim 10, for preparing a composition comprising bone or cartilage cell lineages, comprising the step of culturing a composition comprising iPSM cells in the presence of an efficient amount of at least one or more of the differentiation factors selected from the group consisting of retinoic acid, Wnt, Hedgehog, pTHRP, TGF, BMP families of growth factors, dexamethasone, ascorbic acid, vitamin D3, and b-glycerophosphate.

14. A composition comprising skeletal, bone, cartilage or dermal cells or their progenitors, obtainable by the method according to claim 10.

15. (canceled)

16. The method of claim 2, wherein said primary cells are human cells such.

17. The method of claim 16, wherein said human cells are human fibroblasts.

18. The method of claim 4, wherein said biomarkers characteristic of presomitic mesoderm cells are Msgn1 and/or Tbx6.

19. The method of claim 6, wherein said protein involved in retinoic acid catabolism is Cyp26.

20. The method of claim 9, wherein said biomarker characteristic of presomitic mesoderm cells is an Msgn1 gene product.

21. A method of performing regenerative cell therapy in a patient in need thereof, comprising
administering to the patient a composition comprising iPSM cells obtained by
a) providing target cells to be reprogrammed, and,
b) culturing said target cells under appropriate conditions for reprogramming said cells into iPSM cells, wherein said appropriate conditions comprises comprise increasing expression of at least Brachyury transcription factor in said cells.

22. A method of treating a muscle genetic disease in a patient in need thereof, comprising
administering to the patient a composition comprising iPSM cells obtained by
a) providing target cells to be reprogrammed, and,
b) culturing said target cells under appropriate conditions for reprogramming said cells into iPSM cells, wherein said appropriate conditions comprises comprise increasing expression of at least Brachyury transcription factor in said cells.

23. The method of claim 22, wherein said muscle genetic disease is Duchenne muscular dystrophy.

24. A method of treating joint, cartilage or bone damage in a patient in need thereof, comprising
administering to the patient a composition comprising iPSM cells obtained by
a) providing target cells to be reprogrammed, and,
b) culturing said target cells under appropriate conditions for reprogramming said cells into iPSM cells, wherein said appropriate conditions comprises comprise increasing expression of at least Brachyury transcription factor in said cells.


SEQUENCE LISTING (9 sequences)

SEQ ID NO: 1 - protein, 435 aa, Homo sapiens
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 2 - protein, 436 aa, Mus musculus
MSSPGTESAGKSLQYRVDHLLSAVESELQAGSEKGDPTERELRVGLEESELWLRFKELTNEMIVTKNGRRMFPVLKVNVSGLDPNAMYSFLLDFVTADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERNDHKDVMEEPGDCQQPGYSQWGWLVPGAGTLCPPASSHPQFGGSLSLPSTHGCERYPALRNHRSSPYPSPYAHRNSSPTYADNSSACLSMLQSHDNWSSLGVPGHTSMLPVSHNASPPTGSSQYPSLWSVSNGTITPGSQTAGVSNGLGAQFFRGSPAHYTPLTHTVSAATSSSSGSPMYEGAATVTDISDSQYDTAQSLLIASWTPVSPPSM

SEQ ID NO: 3 - DNA, 1308 bp, Homo sapiens
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatgggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 4 - DNA, 1311 bp, Mus musculus
atgagctcgccgggcacagagagcgcagggaagagcctgcagtaccgagtggaccacctgctcagcgccgtggagagcgagctgcaggcgggcagcgagaagggagaccccaccgaacgcgaactgcgagtgggcctggaggagagcgagctgtggctgcgcttcaaggagctaactaacgagatgattgtgaccaagaacggcaggaggatgttcccggtgctgaaggtaaatgtgtcaggcctggaccccaatgccatgtactctttcttgctggacttcgtgacggctgacaaccaccgctggaaatatgtgaacggggagtgggtacctgggggcaaaccagagcctcaggcgcccagctgcgtctacatccacccagactcgcccaattttggggcccactggatgaaggcgcctgtgtctttcagcaaagtcaaactcaccaacaagctcaatggagggggacagatcatgttaaactccttgcataagtatgaacctcggattcacatcgtgagagttgggggcccgcaacgcatgatcaccagccactgctttcccgagacccagttcatagctgtgactgcctaccagaatgaggagattacagcccttaaaattaaatacaacccatttgctaaagccttccttgatgccaaagaaagaaacgaccacaaagatgtaatggaggaaccgggggactgccagcagccggggtattcccaatgggggtggcttgttcctggtgctggcaccctctgcccgcctgccagctcccaccctcagtttggaggctcgctctctctcccctccacacacggctgtgagaggtacccagctctaaggaaccaccggtcatcgccctaccccagcccctatgctcatcggaacagctctccaacctatgcggacaattcatctgcttgtctgtccatgctgcagtcccatgataactggtctagcctcggagtgcctggccacaccagcatgctgcctgtgagtcataacgccagcccacctactggctctagccagtatcccagtctctggtctgtgagcaatggtaccatcaccccaggctcccagacagctggggtgtccaacgggctgggagctcagttctttcgaggctcccctgcacattacacaccactgacgcacacggtctcagctgccacgtcctcgtcttctggttctccgatgtatgaaggggctgctacagtcacagacatttctgacagccagtatgacacggcccaaagcctcctcatagcctcgtggacacctgtgtcacccccatctatgtga

SEQ ID NO: 5 - DNA, 1233 bp, Homo sapiens
atggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccatcaggatcctggccagctagctag

SEQ ID NO: 6 - DNA, 1212 bp, Mus musculus
atggccagcaatagcagttcctgcccaacacctgggggcgggcacctcaatgggtacccagtacccccctacgccttcttctttccccctatgctgggtggactctccccacccggcgctctcaccagcctccagcaccagcttccagtcagtggttacagcacaccgtccccagccaccatcgagacccagagcagcagttccgaagagatagtacccagccctccctcaccaccgcccctgccccgcatctacaagccttgctttgtttgtcaagacaaatcatccggctaccactatggggtcagcgcctgtgagggctgtaagggcttcttccgacgaagcatccagaagaacatggtgtatacgtgtcaccgggacaagaactgcatcatcaacaaggtgacccggaaccgctgccagtactgccggctgcagaaatgtttcgacgtgggcatgtccaaggagtcggtgcgaaacgatcgaaacaaaaagaagaaagaggcacccaagcccgagtgctcagagagctacacgctgacgcctgaggtgggcgagctcattgagaaggttcgcaaagcgcaccaggagaccttcccggccctctgccagctgggcaagtacactacgaacaacagctcagaacaacgagtctccctggacattgacctctgggacaagttcagtgaactctccaccaagtgcatcattaagactgtggagttcgccaagcagcttcccggcttcaccaccctcaccatcgccgaccagatcaccctcctcaaggctgcctgcctggatatcctgattctgcgaatctgcacgcggtacacgcctgagcaagacacaatgaccttctcagatggactgaccctgaaccggactcagatgcacaacgctggctttggccccctcaccgacttggtctttgccttcgccaaccagctgctgcccctggagatggacgatgctgagactggactgctcagtgccatctgcctcatctgtggagaccgacaggacctggagcagccagacaaggtggacatgctgcaagagccgctgctggaagcactgaaagtctacgtccggaaacggaggcccagccgaccccacatgttccccaagatgctgatgaagatcacagaccttcggagcatcagcgccaagggagctgaacgggtgatcacattgaagatggagatcccataa

SEQ ID NO: 7 - DNA, 582 bp, Homo sapiens
atggacaacctgcgcgagactttcctcagcctcgaggatggcttgggctcctctgacagccctggcctgctgtcttcctgggactggaaggacagggcagggccctttgagctgaatcaggcctccccctctcagagcctttccccggctccatcgctggaatcctattcttcttctccctgtccagctgtggctgggctgccctgtgagcacggcggggccagcagtgggggcagcgaaggctgcagtgtcggtggggccagtggcctggtagaggtggactacaatatgttagctttccagcccacccaccttcagggcggtggtggccccaaggcccagaagggcaccaaagtcaggatgtctgtccagcggaggcggaaagccagcgagagggagaagctcaggatgaggaccttggcagatgccctgcacaccctccggaattacctgccacctgtctacagccagagaggccagcctctcaccaagatccagacactcaagtacaccatcaagtacatcggggaactcacagacctccttaaccgcggcagagagcccagagcccagagcgcgtga

SEQ ID NO: 8 - DNA, 567 bp, Mus musculus
atggacaacctgggtgagaccttcctcagcctggaggatggcctggactcttctgacaccgctggtctgctggcctcctgggactggaaaagcagagccaggcccttggagctggtccaggagtcccccactcaaagcctctccccagctccttctctggagtcctactctgaggtcgcactgccctgcgggcacagtggggccagcacaggaggcagcgatggctacggcagtcacgaggctgccggcttagtcgagctggattacagcatgttggcttttcaacctccctatctacacactgctggtggcctcaaaggccagaaaggcagcaaagtcaagatgtctgtccagcggagacggaaggccagcgagagagagaaactcaggatgcggaccttagccgatgccctccacacgctccggaattacctgccgcctgtctacagccagagaggccaaccgctcaccaagatccagacactcaagtacaccatcaagtacatcggggaactcacagacctcctcaacagcagcgggagagagcccaggccacagagtgtgtga

SEQ ID NO: 9 - DNA, 1308 bp, Homo sapiens; identical to SEQ ID NO: 3